(19) United States
(12) Patent Application Publication
(10) Pub. No.: US 2023/0406947 A1
(43) Pub. Date: Dec. 21, 2023

(54) LIGHT INHIBITORS FOR SCLERODERMA AND SKIN FIBROTIC DISEASE TREATMENT

(71) Applicant: La Jolla Institute for Allergy and Immunology, La Jolla, CA (US)

(72) Inventors: Michael CROFT, La Jolla, CA (US); Rana HERRO, La Jolla, CA (US)

(73) Assignee: La Jolla Institute for Allergy and Immunology, La Jolla, CA (US)

(21) Appl. No.: 18/094,151

(22) Filed: Jan. 6, 2023

Related U.S. Application Data
(63) Continuation of application No. 15/549,088, filed on Aug. 4, 2017, now abandoned.
(60) Provisional application No. 62/112,571, filed on Feb. 5, 2015.

(51) Int. Cl. C07K 16/28; A61K 39/395; A61K 45/06

(52) U.S. Cl. CPC C07K 16/2878; A61K 39/3955; A61K 45/06; A61K 2039/505

(57) ABSTRACT

Methods of treating inflammatory conditions, disease and disorders of skin are provided. Methods include, for example, contacting or administering a sufficient amount of a LIGHT inhibitor to a subject to treat skin inflammation, skin fibrosis, or a skin fibrotic disease or disorder such as scleroderma, atopic dermatitis, nephrogenic fibrosing dermopathy, mixed connective tissue disease, scleromyxedema, scleredema, keloid, sclerodactyly, or eosinophilic fasciitis.

### RELATED APPLICATIONS

This application claims the benefit of priority to U.S. provisional application No. 62/112,571, filed Feb. 5, 2015, which application is expressly incorporated herein by reference in its entirety.

### STATEMENT AS TO FEDERALLY SPONSORED RESEARCH

The invention was supported in part by National Institute of Health Grants AI070535 and AI100905. The government has certain rights in the invention.

### INTRODUCTION

Fibrosis and thickening of the skin is a characteristic of several inflammatory and autoimmune diseases including systemic sclerosis (SSc) or scleroderma, and atopic dermatitis (Boin and Wigley, 2009; Rosenbloom et al., 2010; Wynn and Ramalingam, 2012; Yamamoto, 2009). Current treatments involve non-selective immunotherapy with corticosteroids, D-penicillamine, methotrexate, or cyclophosphamide, but defining new targets for intervention of fibrosis in the skin is important. Fibrosis is a feature that is shared with other diseases such as severe asthma, and autoimmune diseases like RA, Crohn's disease, and SLE, but whether there are common molecules that promote clinical symptoms across these syndromes is not clear. Recent studies of Thymic Stromal LymphoPoietin (TSLP) have suggested it may be central to fibrosis in a number of different tissues (Comeau and Ziegler, 2010; Yoo et al., 2005b; Ziegler and Artis, 2010). Transgenic expression of TSLP in keratinocytes resulted in spontaneous development of features of atopic dermatitis or scleroderma, and transgenic expression of TSLP in lung epithelial cells promoted lung fibrosis characteristic of severe asthma (Yoo et al., 2005b; Zhou et al., 2005). Furthermore, human keratinocytes in the skin of patients with atopic dermatitis or systemic sclerosis express high levels of TSLP, and elevated TSLP is also seen in asthmatics (Alysandratos et al., 2010; Lee et al., 2010; Sano et al., 2013; Shikotra et al., 2012; Yao et al., 2013; Ying et al., 2008). TNF superfamily member 14 (TNFSF14), known as LIGHT and CD258 (Ware, 2005, 2009), strongly promoted fibrosis in the lungs of mice chronically challenged with allergen (Doherty et al., 2011).

In the studies described herein, LIGHT can promote fibrotic features in the skin. Direct injection of recombinant LIGHT into naïve mice promoted dermal and epidermal thickening. Additionally, fibrotic features in the skin were abrogated in LIGHT-deficient mice treated with bleomycin in a mouse model that produces symptoms reminiscent of those exhibited in scleroderma (Yamamoto, 2006; Yamamoto and Nishioka, 2005). Importantly, the action of LIGHT was dependent on activity of TSLP, and also TGF-β, and stimulation of keratinocytes with recombinant LIGHT promoted TSLP expression. These data indicate that LIGHT may be central to fibrosis and may be amenable to clinical targeting in diseases of the skin.

LIGHT (TNFSF14, p30 polypeptide) is a protein expressed on activated CD4/CD8 T cells, dendritic cells (DCs), monocytes, and natural killer cells (NK). The binding of LIGHT to herpes virus entry mediator (HVEM), which is expressed on resting T cells, DCs, and monocytes, or the lymphotoxin beta receptor (LTβR), which is expressed on DCs and stromal cells, promotes T cell activation, proliferation, and cytokine production. Studies have determined that LIGHT deficient animals have no significant abnormalities in the development of lymphoid organs and lymphocytes.

### SUMMARY

As disclosed herein, injection of recombinant soluble LIGHT into naïve mice, either subcutaneously or systemically, promoted collagen deposition in the skin, and dermal and epidermal thickening. This replicated the activity of bleomycin, an antibiotic that has been previously used in models of systemic sclerosis or scleroderma in mice. Moreover skin fibrosis induced by bleomycin was dependent on endogenous LIGHT activity. The action of LIGHT in vivo was mediated via both of its receptors, HVEM and LTβR, and was dependent on the innate cytokine TSLP and TGF-β. Furthermore, HVEM and LTβR were expressed on human epidermal keratinocytes, and LIGHT can directly promote TSLP expression in these cells. The data reveal activity of LIGHT on keratinocytes and that LIGHT may be an important mediator of skin inflammation and fibrosis in diseases such as scleroderma or atopic dermatitis.

Inhibiting, suppressing or blocking LIGHT from interacting with its receptors, HVEM or LTβR, can be used as an anti-inflammatory, for example, to inhibit or suppress skin inflammation, as well as treat skin fibrosis, scleroderma, atopic dermatitis, and other skin fibrotic diseases and disorders. Accordingly, the invention is based at least in part on the finding that LIGHT (P30 polypeptide) is a viable target for treating skin inflammation, skin fibrosis, or skin fibrotic diseases and disorders, such as scleroderma and atopic dermatitis.

In accordance with the invention, there are provided, methods of reducing or inhibiting skin inflammation, skin fibrosis, or a skin fibrotic disease or disorder. In one embodiment, a method includes contacting or administering a sufficient amount of an inhibitor of LIGHT (p30 polypeptide) to a subject in need thereof to reduce skin inflammation, skin fibrosis, or a skin fibrotic disease or disorder in the subject.

In accordance with the invention, there are also provided, methods of treating scleroderma, atopic dermatitis, nephrogenic fibrosing dermopathy, mixed connective tissue disease, scleromyxedema, scleredema, keloid, sclerodactyly, or eosinophilic fasciitis. In one embodiment, a method includes contacting or administering a sufficient amount of an inhibitor of LIGHT (p30 polypeptide) to a subject in need thereof to treat scleroderma, atopic dermatitis, nephrogenic fibrosing dermopathy, mixed connective tissue disease, scleromyxedema, scleredema, keloid, sclerodactyly, or eosinophilic fasciitis.

LIGHT inhibitors include, for example, molecules that bind to LIGHT and inhibit LIGHT binding or interaction with HVEM. LIGHT inhibitors also include molecules that bind to LIGHT and inhibit LIGHT binding or interaction with LTβR. LIGHT inhibitors further include molecules that bind to HVEM and inhibit LIGHT binding or interaction with HVEM. LIGHT inhibitors additionally include molecules that bind to LTβR and inhibit LIGHT binding or interaction with LTβR. LIGHT inhibitors moreover include prodrugs of the foregoing.

Invention methods include contact or administration, in vitro, ex vivo or in vivo (e.g., to a subject in need of treatment). In various embodiments, skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis or any symptom thereof, is reduced, decreased, inhibited, delayed, halted, or prevented in the subject, locally, or regionally in an area (region) of the subject. In particular aspects, a symptom is reduced, decreased, inhibited, delayed, halted, or prevented in skin. In another aspect, a method reduces, decreases, inhibits, delays, halts, or prevents skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis, or any symptom thereof. In yet another embodiment, contacting or administration in vivo is in a subject that has previously experienced skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis, or any symptom thereof, or is in need of inhibited or reduced skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitisor any symptom thereof.

In accordance with the invention, there are also provided, methods of inhibiting, reducing or decreasing progression, severity, frequency, duration or probability of one or more symptoms caused by or associated with skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. In one embodiment, a method includes administering to a subject an amount of a LIGHT inhibitor sufficient to inhibit, reduce or decrease progression, severity, frequency, duration or probability of a symptom associated with skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. In various aspects, skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis, is caused by an allergen or is not caused by an allergen.

Non-limiting symptoms of skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis include, for example, skin inflammation or tissue damage; hardening or tightening of patches of skin; thickening of the epidermis or dermis; skin tenderness; skin itching; skin rash; heightened response or sensitivity of skin to cold or hot temperatures; or numbness, pain or color changes in the fingers or toes. Further symptoms, include, for example, infiltration of eosinophils in skin, leukocyte infiltration of skin, inflammation of skin, or increased cytokine production.

Invention treatment methods include providing a given subject with an objective or subjective improvement of the condition, disorder or disease, a symptom caused by or associated with the condition, disorder or disease, or the probability or susceptibility of a subject to the condition or a symptom caused by or associated with the condition, disorder or disease. In various embodiments, treatment reduces, decreases, inhibits, delays, eliminates or prevents the probability, susceptibility, severity, frequency, or duration of one or more symptoms caused by or associated with the condition, disorder or disease. In a particular aspect, a method reduces the probability, severity, frequency, duration or delays, halts, or prevents skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. In additional aspects, treatment improves or increases skin elasticity. In further aspects, a treatment improves, reduces or inhibits skin-tightening or hardening, or thickening of epidermis or dermis.

Candidate subjects for methods of the invention include mammals, such as humans. Candidate subjects for methods of the invention also include subjects that are in need of treatment, e.g., any subject that may benefit from a treatment. Candidate subjects for methods of the invention therefore include subjects that have or are at risk of having skin inflammation, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis, or any symptom thereof.

Methods of the invention can be practiced by administration or contact with any dose amount, frequency, delivery route or timing of a LIGHT inhibitor. In particular embodiments, a subject is administered or contacted a LIGHT inhibitor one, two, three, four or more times hourly, daily, bi-weekly, weekly, monthly or annually. In additional embodiments, an amount administered is about 0.00001 mg/kg, to about 10,000 mg/kg, about 0.0001 mg/kg, to about 1000 mg/kg, about 0.001 mg/kg, to about 100 mg/kg, about 0.01 mg/kg, to about 10 mg/kg, about 0.1 mg/kg, to about 1 mg/kg body weight, one, two, three, four, or more times per hour, day, bi-weekly, week, month or annually. In further embodiments, the amount administered is less than about 0.00001 mg/kg, one, two, three, four, or more times per hour, day, bi-weekly, week, month or annually. In particular aspects, the amount is administered substantially contemporaneously with, or within about 1-60 minutes, hours, or days of the onset of a symptom caused by or associated with skin inflammation, skin fibrosis, scleroderma, or a skin fibrotic disease or disorder.

Methods of the invention include routes of contact or administration of LIGHT inhibitor locally, regionally and systemically. In a particular embodiment, a LIGHT inhibitor is administered topically or systemically to achieve delivery to skin or dermis.

Methods of the invention can be practiced in conjunction with one or more other treatment protocols or therapeutic regimens. In a particular embodiment, a method includes contacting or administering a second agent or drug to the subject prior to, with or following contacting or administering LIGHT inhibitor. In particular aspects, a second agent or drug includes an anti-skin inflammation, anti-skin fibrosis, anti-scleroderma, or an anti-skin fibrotic disease or disorder drug or agent.

Invention compositions can be formulated as appropriate for practice of the methods. In one embodiment, a composition includes a LIGHT inhibitor, and a pharmaceutically acceptable carrier. In a particular aspect, the carrier is a physiologically acceptable gas, liquid, dry powder or an aerosol. In an additional particular aspect, the carrier is a capable of contact with skin or dermis, or traversing skin or dermis. In a further particular aspect, the carrier is lipophilic or non-lipophilic.

### BRIEF DESCRIPTION OF DRAWINGS

FIG. 1 shows that recombinant LIGHT induces skin fibrosis in naïve mice. Naïve WT mice were injected with 10 μg of soluble rmLIGHT or PBS alone, administered subcutaneously (SC, on the back between the ears) or intratracheally (IT) on days 1 and 2. Skin inflammation and fibrosis were assessed one day later. (a-b) Skin sections (magnification 20×) were stained with Masson's trichrome and an antibody to aSMA (a), and quantified for epidermal and dermal thickening (b). Values from individual tissues from 5 mice. Data are representative of 6 studies. ***, p<0.01; ****, p<0.001. (c) qPCR analysis was performed on skin samples and mRNA expression of collagen, aSMA, and TGF-β calculated relative to L32. Values are mean±SEM from 2 to 4 mice per condition. (d) Total cells, dendritic cells (DC), neutrophils, macrophages, and T cells, were enumerated in skin samples. Values are from individual tissues from 3-6 mice.

FIG. 2 shows that LIGHT-deficient mice exhibit decreased skin fibrosis induced by bleomycin. WT and LIGHT^{-/-} mice were administered 0.2 U bleomycin/mouse. Mice were sacrificed on day 7. (a) Skin fibrosis was assessed by analyzing trichrome (mag. 10×) and aSMA stained sections (mag. 20×). (b) Dermal and epidermal thickness was quantitated. Values from individual tissues from 6 mice. Data are representative of 6 studies. ***, p<0.01; ****, p<0.001. (c) Total cells, dendritic cells (DC), neutrophils, macrophages, and T cells, were enumerated in skin samples. Values are from individual tissues from 5 mice.

FIG. 3 shows that HVEM and LTβR differentially contribute to skin fibrosis. WT and HVEM^{-/-} mice were compared to WT mice treated with 200 μg neutralizing anti-LTβR. Mice were either administered 10 μg rmLIGHT SC or IT on days 1 and 2 before sacrificing the mice at day 3, or were given 0.2 U bleomycin/mouse and sacrificed on day 7. Fibrotic activity in the skin was assessed as before. (a) Trichrome staining. (b) aSMA staining (mag. 20×). Quantification from individual tissues from 6 mice. Data representative of 2 studies. ***, p<0.01; ****, p<0.001.

FIG. 4 shows that LIGHT promotes skin fibrosis dependent on TSLP. (a) WT mice were treated SC or IT with 10 μg of rmLIGHT or PBS on days 1 and 2. TSLP expression was measured in the skin by IHC (green, mag. 20×) or PCR after 3 days. (b) WT, LIGHT^{-/-} and TSLPR^{-/-} mice were compared for skin fibrosis after treatment with bleomycin. WT mice were assessed at day 7, whereas gene-deficient mice were analyzed on day 14. (c) WT and LIGHT^{-/-} mice were treated with bleomycin and skin was assessed for TSLP expression on day 7 by IHC (green) (mag. 20×). (d-e) WT and TSLPR^{-/-} mice were administered 10 μg of rmLIGHT on days 1 and 2. Skin fibrosis (d) and cellular infiltration (e) was assessed as before with values representing 3 to 6 mice per group. All data are representative of 4 studies.

FIG. 5 shows that TGF-β is required for LIGHT driven skin fibrosis. (a) WT, LIGHT^{-/-}, HVEM^{-/-}, and WT mice treated with anti-LTβR, were challenged with intratracheal bleomycin, and after 7 days skin expression of TGF-β1 mRNA was assessed. Values are mean±SEM of 3 to 4 mice per group. (b) WT mice, treated with control IgG or anti-TGF-β, were administered 10 μg of rmLIGHT on days 1 and 2. Skin fibrosis and TSLP expression was assessed as before on day 3. Data are representative of 3 individual mice analyzed per group.

FIG. 6 shows that LIGHT induces TSLP in keratinocytes. (a) Human epidermal keratinocytes (HEKn) and mouse PAM212 keratinocytes were stained for LTβR and HVEM expression by flow cytometry. Isotype control in red. (b) HEKn and PAM212 cells were stimulated in vitro with 20 ng/ml of soluble rhLIGHT or rmLIGHT for 72 h. TSLP expression was evaluated by IHC (green and red stain) and mRNA levels by qPCR. Values are mean±SEM of triplicate samples per condition. (c) PAM212 cells were stimulated as in (b) in the presence of control IgG or anti-TGF-β, and TSLP expression analyzed by IHC. All data are representative of 4 studies.

FIG. 7 shows LIGHT-deficiency decreases atopic dermatitis induced by house dust mite (HDM) allergen. 7a) Atopic dermatitis was induced in WT and LIGHT-deficient mice by repeated epicutaneous treatments with 10 μg of HDM extract and 500 ng of SEB per mouse each round. Two epicutaneous treatments lasting 4 days each were performed, and mice were allowed to itch and scratch freely for 4 days in between allergen challenges before being euthanized at day 14. Mice exposed to PBS rather than allergen were used as controls. Collagen deposition measured by Masson trichrome stain, cellular infiltration assessed by H&E stain, and expression of TSLP, Perisotin and alpha smooth muscle actin evaluated by Immunofluorescence, was determined in skin biopsies. 7b) Skin lesions were scored for the severity of each of the four parameters: redness, bleeding, eruption, and scaling, using the following scale 0-no symptoms 1-mild 2-intermediate 3-severe. 7c) Total cell counts were assessed by flow cytometry on punch biopsies of the skin after digestion. Data show LIGHT-deficient animals were protected from development of atopic dermatitis.

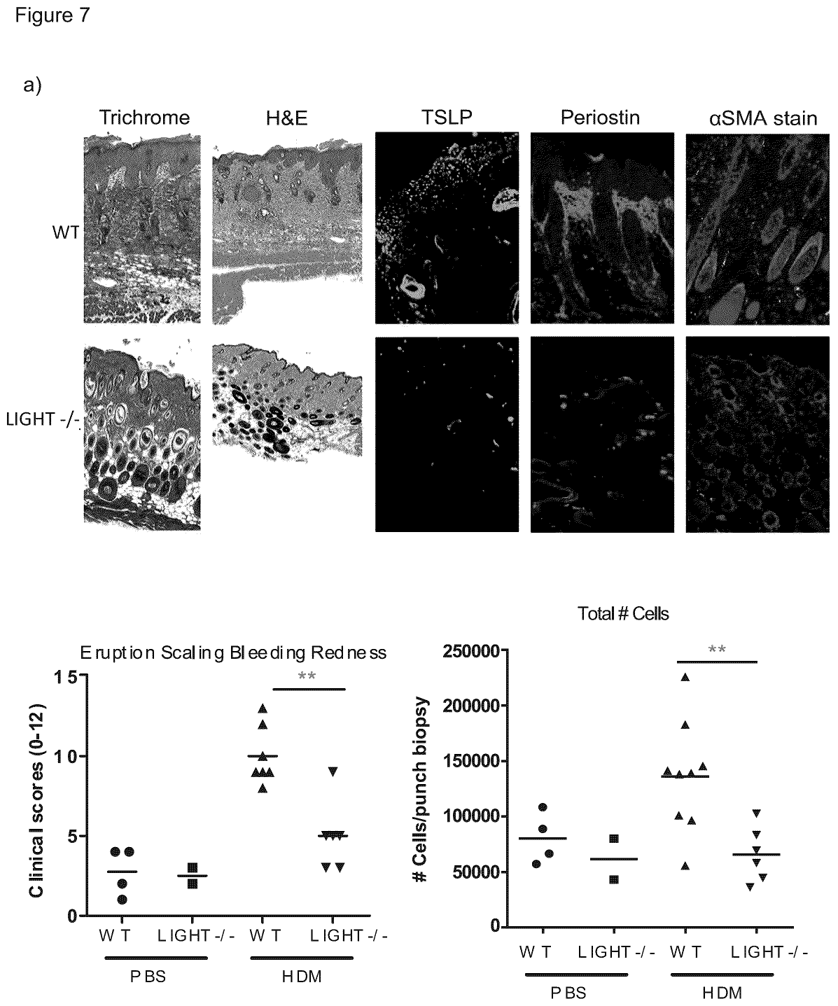

FIG. 8 shows conditional deletion of HVEM in keratinocytes abrogates HDM-driven skin inflammation. 8a) Conditional knockout mice bearing a specific deletion of HVEM in keratinocytes (K14-cre HVEM^{f/f}) and their littermate controls (K14-cre HVEM^{+/+}) were treated epicutaneously with two rounds of HDM/SEB to induce atopic dermatitis-like skin inflammation. At day 14, mice were euthanized and skin biopsies were stained with Masson trichrome to evaluate collagen deposition and H&E to determine the ratio of cellular infiltration in the skin. Skin lesions were scored for the severity of 4 clinical symptoms (eruption, scaling, bleeding and redness) with a comparison to LIGHT-deficient mice. 8b) K14-cre HVEM^{+/+} and K14-cre HVEM^{f/f} mice were challenged with 0.2 U of Bleomycin to induce scleroderma-like skin inflammation. Masson Trichrome stain was performed on skin biopsies at day 7 to evaluate collagen deposition. Data show that mice deficient in HVEM in keratinocytes are protected from development of skin inflammation similar to that seen in atopic dermatitis and scleroderma.

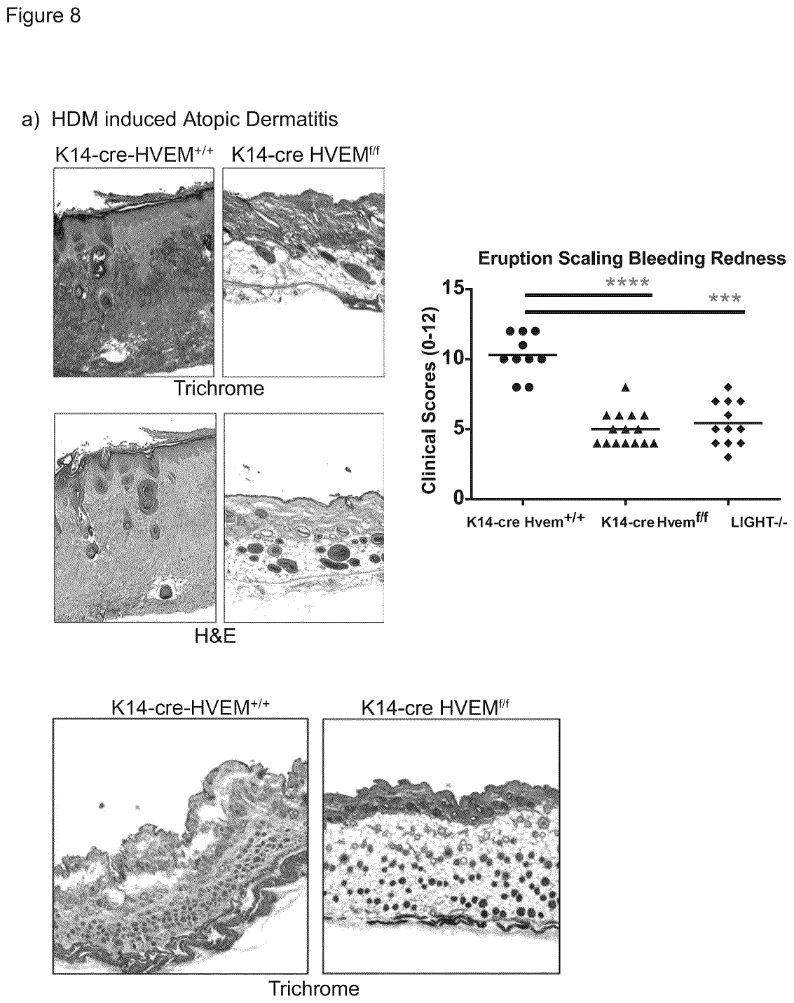

FIG. 9 shows blocking HVEM-LIGHT interactions before or after disease onset reduces HDM-driven Atopic dermatitis. Atopic dermatitis was induced in WT mice using 2 rounds of HDM/SEB epicutaneous exposure. Mice were treated with an antagonistic antibody to HVEM that neutralizes specifically its interaction with LIGHT (anti-HVEM clone LH1) or an Isotype control IgG using two regimens: 1-prophylactic, 100 μg given i.p. 24 h before the first allergen challenge and every other day until the end of the experiment (day 14), 2-therapeutic, 200 μg given i.p. starting on day 5 and given every other day until day 14. Collagen deposition was assessed with Masson trichrome stain and cellular infiltration with H&E stain, performed on skin biopsies. Skin lesions were scored visually by evaluating the severity of 4 clinical symptoms (eruption, scaling, bleeding and redness). Data show neutralizing HVEM-LIGHT interactions, prophylatically or therapeutically, inhibits atopic dermatitis symptoms.

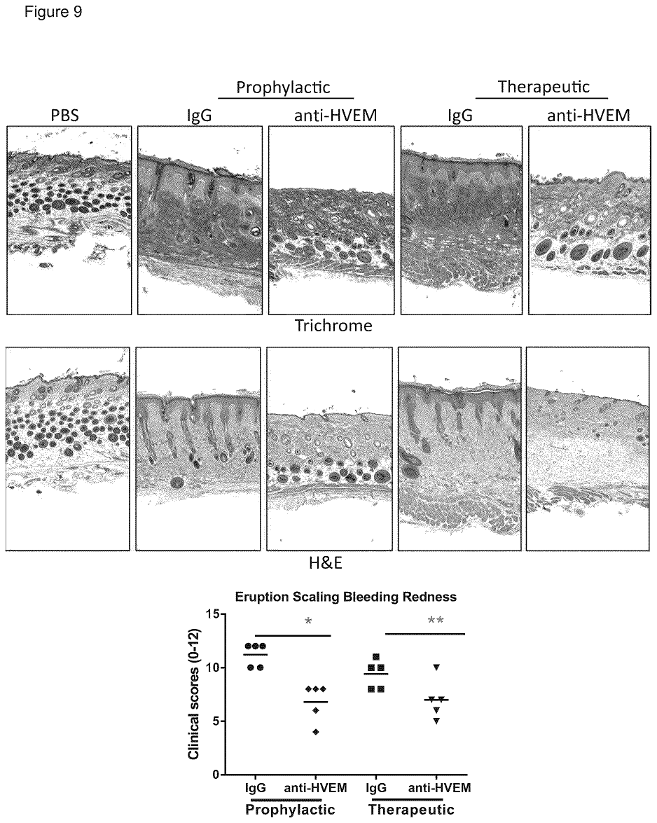

### DETAILED DESCRIPTION

The invention provides methods of reducing or inhibiting skin inflammation, skin fibrosis, skin fibrosis or a skin fibrotic diseases and disorders, scleroderma, atopic dermatitis, nephrogenic fibrosing dermopathy, mixed connective tissue disease, scleromyxedema, scleredema, keloid, sclerodactyly, or eosinophilic fasciitis. The invention also provides methods of treating skin inflammation, skin fibrosis, skin fibrotic diseases and disorders, scleroderma, atopic dermatitis, nephrogenic fibrosing dermopathy, mixed connective tissue disease, scleromyxedema, scleredema, keloid, sclerodactyly, or eosinophilic fasciitis. In various embodiments, a method includes contacting or administering a sufficient amount of an inhibitor of LIGHT (p30 polypeptide) to a subject to reduce or inhibit skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis, to treat skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis.

The term “an inhibitor of LIGHT,” means a molecule that directly or indirectly inhibits binding of LIGHT (p30 polypeptide) to HVEM or to LTβR. Inhibitors therefore include molecules that bind to LIGHT as well as molecules that bind to a LIGHT receptor or target. Since LIGHT (p30 polypeptide) can bind to a variety of receptors and targets, such as HVEM and LTβR, LIGHT (p30 polypeptide) inhibitors therefore include molecules that bind to LIGHT (p30 polypeptide), molecules that bind to HVEM, as well as molecules that bind to LTβR, which can thereby inhibit binding of LIGHT to HVEM, binding of LIGHT to LTβR, etc., either directly or indirectly.

A non-limiting representative example of human LIGHT (p30 polypeptide) sequence (SEQ ID NO:1; the amino acid residues of the transmembrane domain are shaded, and the amino acid residues of the extracellular domain are underlined) target for an inhibitor is as set forth below:

MEESVVRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEEVVVRVLDERLVRLRDGTRSYFGAFMV

A non-limiting representative example of human HVEM (herpesvirus entry mediator) sequence target for an inhibitor, also referred to as tumor necrosis factor receptor superfamily, member 14 (TNFRSF14) is as set forth below (SEQ ID NO:2):

MEPPGDWGPPPWRSTPKTDVLRLVLYLTELGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH

A non-limiting representative example of human LTβR sequence target for an inhibitor, is as set forth below (SEQ ID NO:3):

MLLPWATSAPGLAWGPLVLGLFGLLAASQPQAVPPYASENQTCRDQEKEY
YEPQHRICCSRCPPGTYVSAKCSRIRDTVCATCAENSYNEHWNYLTICQL
CRPCDPVMGLEEIAPCTSKRKTQCRCQPGMFCAAWALECTHCELLSDCPP
GTEAELKDEVGKGNNHCVPCKAGHFQNTSSPSARCQPHTRCENQGLVEAA
PGTAQSDTTCKNPLEPLPPEMSGTMLMLAVLLPLAFFLLLATVESCIWKS
HPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGINGIHV
TGGSMTITGNIYIYNGPVLGGPPGPGDLPATPEPPYPIPEEGDPGPPGLS
TPHQEDGKAWHLAETEHCGATPSNRGPRNQFITHD

Exemplary LIGHT inhibitors include, for example, small organic compounds (e.g., drugs), polypeptide sequences such as antibodies and antibody subsequences that bind to LIGHT (p30 polypeptide), HVEM (herpesvirus entry mediator) or LTβR (lymphotoxin beta receptor). Additional exemplary LIGHT inhibitors include, for example, a LIGHT, HVEM or LTβR (lymphotoxin beta receptor) polypeptide subsequence, variant sequence, chimeric sequence or dominant negative sequence (e.g., soluble forms of LIGHT, HVEM or LTβR). Further exemplary LIGHT inhibitors include, for example, chimeric sequences, such as a fusion of a LIGHT, HVEM or LTβR polypeptide sequence (e.g., soluble forms of LIGHT, HVEM or LTβR) and an immunoglobulin (Ig) sequence.

Exemplary LIGHT antibodies include, for example, antibodies that bind to human LIGHT. Non-limiting examples of commercially available antibodies that bind to human LIGHT include clone T5-39 (BioLegend, San Diego, CA), clone 115520 (R&D Systems, Minneapolis, MN), clones A-20 and C-20 (Santa Cruz Biotech, Santa Cruz, CA), and clone 4E3 (Novus Biologicals, Inc., Littleton, CO).

Antibodies include mammalian, human, humanized, humaneered or primatized forms of heavy or light chain, V_{H} and V_{L}, respectively, immunoglobulin (Ig) molecules. An “antibody” means any monoclonal or polyclonal immunoglobulin molecule, such as IgM, IgG, IgA, IgE, IgD, and any subclass thereof, which includes intact immunoglobulin molecules, two full length heavy chains linked by disulfide bonds to two full length light variable domains, V_{H} and V_{L}, individually or in any combination, as well as subsequences, such as Fab, Fab′, (Fab′)₂, Fv, Fd, scFv and sdFv, unless otherwise expressly stated.

An antibody that binds to LIGHT, HVEM or LTβR antibody means that the antibody has affinity for LIGHT, HVEM or LTβR. “Specific binding” is where the binding is selective between the two referenced molecules. Thus, specific binding of an antibody for LIGHT, HVEM or LTβR is that which is selective for an epitope present in LIGHT, HVEM or LTβR. Typically, specific binding can be distinguished from non-specific when the dissociation constant (K_{D}) is less than about 1×10⁻⁵ M or less than about 1×10⁻⁶ M or 1×10⁻⁷ M. Selective binding can be distinguished from non-selective binding using assays known in the art (e.g., immunoprecipitation, ELISA, Western blotting) with appropriate controls.

Monoclonal antibodies are made by methods known in the art (Kohler et al., Nature, 256:495(1975); and Harlow and Lane, Using Antibodies: A Laboratory Manual, Cold Spring Harbor Laboratory, 1999). Briefly, monoclonal antibodies can be obtained by injecting mice with antigen. The polypeptide or peptide used to immunize an animal may be derived from translated DNA or chemically synthesized and conjugated to a carrier protein. Commonly used carriers which are chemically coupled to the immunizing peptide include, for example, keyhole limpet hemocyanin (KLH), thyroglobulin, bovine serum albumin (BSA), and tetanus toxoid. Antibody production is verified by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from hybridoma cultures. Monoclonal antibodies can be isolated and purified from hybridoma cultures by a variety of established techniques which include, for example, affinity chromatography with Protein-A Sepharose, size-exclusion chromatography, and ion-exchange chromatography (see e.g., Coligan et al., Current Protocols in Immunology sections 2.7.1-2.7.12 and sections 2.9.1-2.9.3; and Barnes et al., “Methods in Molecular Biology,” 10:79-104, Humana Press (1992)).

A “human antibody” means that the amino acid sequence of the antibody is fully human, i.e., human heavy and light chain variable and constant regions. The antibody amino acids are coded for in the human DNA antibody sequences or exist in a human antibody. Fully human antibodies can be made by human antibody transgenic or transchromosomic animals, such as mice, or by isolation from human antibody producing cell lines (e.g., B cells) by recombinant DNA methodology known to the skilled artisan, such as gene cloning by reverse transcriptase polymerase chain reaction (RT-PCR). An antibody that is non-human may be made fully human by substituting non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); Chothia and Lesk, J. Mol. Biol. (1987) 186:651; Padlan Mol. Immunol. (1994) 31:169; and Padlan Mol. Immunol. (1991) 28:489). Methods of producing human antibodies are also described, for example, in WO 02/43478 and WO 02/092812.

The term “humanized,” when used in reference to an antibody, means that the antibody sequence has non-human amino acid residues of one or more complementarity determining regions (CDRs) that specifically bind to the antigen in an acceptor human immunoglobulin molecule, and one or more human amino acid residues in the framework region (FR) that flank the CDRs. Any mouse, rat, guinea pig, goat, non-human primate (e.g., ape, chimpanzee, macaque, orangutan, etc.) or other animal antibody may be used as a CDR donor for producing humanized antibody. Human framework region residues can be replaced with corresponding non-human residues (e.g., from the donor variable region). Residues in the human framework regions can therefore be substituted with a corresponding residue from the non-human CDR donor antibody. A humanized antibody may include residues, which are found neither in the human antibody nor in the donor CDR or framework sequences. The use of antibody components derived from humanized monoclonal antibodies reduces problems associated with the immunogenicity of non-human regions. Methods of producing humanized antibodies are known in the art (see, for example, U.S. Pat. Nos. 5,225,539; 5,530,101, 5,565,332 and 5,585,089; Riechmann et al., (1988) Nature 332:323; EP 239,400; WO91/09967; EP 592,106; EP 519,596; Padlan Molecular Immunol. (1991) 28:489; Studnicka et al., Protein Engineering (1994) 7:805; Singer et al., J. Immunol. (1993) 150:2844; and Roguska et al., Proc. Nat'l. Acad. Sci. USA (1994) 91:969).

The term “humanized,” when used in reference to an antibody, means that the antibody sequence has high affinity for antigen but has a greater number of human germline sequences than a humanized antibody. Typically humaneered antibody has at least 90% or more human germline sequences.

As used herein, the terms “peptide,” “polypeptide” and “protein” are used interchangeably and refer to two or more amino acids covalently linked by an amide bond or non-amide equivalent. Polypeptides include full length native polypeptide, and “modified” forms such as subsequences, variant sequences, fusion/chimeric sequences and dominant-negative sequences.

Peptides include L- and D-isomers, and combinations thereof. Peptides can include modifications typically associated with post-translational processing of proteins, for example, cyclization (e.g., disulfide or amide bond), phosphorylation, glycosylation, carboxylation, ubiquitination, myristylation, or lipidation. Modified peptides can have one or more amino acid residues substituted with another residue, added to the sequence or deleted from the sequence. Specific examples include one or more amino acid substitutions, additions or deletions (e.g., 1-3, 3-5, 5-10, 10-20, or more).

Subsequences and fragments refer to polypeptides having one or more fewer amino acids in comparison to a reference (e.g., native) polypeptide sequence. An antibody subsequence that specifically binds to LIGHT, HVEM or LTβR can retain at least a part of its binding or LIGHT inhibitory or antagonist activity.

A variant peptide can have a sequence with 50%, 60%, 70%, 75%, 80%, 85%, 90%, 95%, 96%, 97%, 98%, or more identity to a reference sequence. Variant sequences include naturally occurring alterations of sequence, due to intra-species polymorphisms or different species, as well as artificially produced alterations of sequence. Sequence homology between species is in the range of about 70-80%. An amino acid substitution is one example of a variant.

A “conservative substitution” is the replacement of one amino acid by a biologically, chemically or structurally similar residue. Biologically similar means that the substitution is compatible with an activity or function of the unsubstituted sequence. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

Peptides synthesized and expressed as fusion proteins have one or more additional domains linked thereto, and are also referred to as chimeric polypeptides. The additional domain(s) may confer an additional function upon the sequence. For example, HVEM-IgG or LTβR-IgG fusion proteins can have LIGHT inhibitory activity.

The term “fusion,” when used in reference to two or more molecules (e.g., polypeptides) means that the molecules are covalently attached. A particular example for attachment of two protein sequences is an amide bond or equivalent. The term “chimeric,” and grammatical variations thereof, when used in reference to a protein, means that the protein is comprised of one or more heterologous amino acid residues from one or more different proteins.

The term “heterologous,” when used in reference to a polypeptide, means that the polypeptide is not normally contiguous with the other polypeptide in its natural environment. Thus, a chimeric polypeptide means that a portion of the polypeptide does not exist fused with the other polypeptide in normal cells. In other words, a chimeric polypeptide is a molecule that does not normally exist in nature, i.e., such a molecule is produced by the hand of man, e.g., artificially produced through recombinant DNA technology.

As used herein, the term “mimetic” refers to a synthetic chemical compound which has substantially the same structural and/or functional characteristics as the reference molecule. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy activity.

Peptide mimetics can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., induce or stabilize a secondary structure, e.g., a beta turn, gamma turn, beta sheet, alpha helix conformation, and the like. For example, a polypeptide can be characterized as a mimetic when one or more of the residues are joined by chemical means other than an amide bond. Individual peptidomimetic residues can be joined by amide bonds, non-natural and non-amide chemical bonds other chemical bonds or coupling means including, for example, glutaraldehyde, N-hydroxysuccinimide esters, bifunctional maleimides, N,N′-dicyclohexylcarbodiimide (DCC) or N,N′-diisopropylcarbodiimide (DIC). Linking groups alternative to the amide bond include, for example, ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide and Backbone Modifications,” Marcel Decker, NY).

Peptides and peptidomimetics can be produced and isolated using a variety of methods known in the art. Full length peptides and fragments (subsequences) can be synthesized using chemical methods known in the art (see, e.g., Caruthers, Nucleic Acids Res. Symp. Ser. (1980) 215; Horn, Nucleic Acids Res. Symp. Ser. (1980) 225; and Banga, A. K., Therapeutic Peptides and Proteins, Formulation, Processing and Delivery Systems (1995) Technomic Publishing Co., Lancaster, PA). Peptide synthesis can be performed using various solid-phase techniques (see, e.g., Roberge, Science (1995) 269:202; Merrifield, Methods Enzymol. (1997) 289:3). Automated synthesis may be achieved, e.g., using a peptide synthesizer.

Individual synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Peptides and peptide mimetics can also be synthesized using combinatorial methodologies. Techniques for generating peptide and peptidomimetic libraries are known, and include, for example, multipin, tea bag, and split-couple-mix techniques (see, for example, al-Obeidi, Mol. Biotechnol. (1998) 9:205; Hruby, Curr. Opin. Chem. Biol. (1997) 1:114; Ostergaard, Mol. Divers. (1997) 3:17; and Ostresh, Methods Enzymol. (1996) 267:220). Modified peptides can be further produced by chemical modification methods (see, e.g., Belousov, Nucleic Acids Res. (1997) 25:3440; Frenkel, Free Radic. Biol. Med. (1995) 19:373; and Blommers, Biochemistry (1994) 33:7886).

Inhibitors of LIGHT therefore include those that can bind selectively as well as those that bind non-selectively to a ligand or target (e.g., LIGHT, HVEM, LTβR, etc.) in solution, in solid phase, in vitro, ex vivo or in vivo. As used herein, the term “selective” when used in reference to a LIGHT inhibitor, means that the inhibitor binds specifically to the target entity (e.g., LIGHT, HVEM, LTβR, etc.) and does not significantly bind to a non-ligand or non-target entity. A non-selective inhibitor means that the inhibitor is not selective for the entity to which it binds, i.e., it cross-reacts with other entities.

LIGHT inhibitors include variants and derivatives that retain at least a part or all of an activity of the non-variant or non-derivatized inhibitor. A particular activity (e.g., antagonist or inhibitory activity) of a LIGHT inhibitor may be less than or greater than the activity of a corresponding non-variant or non-derivatized LIGHT inhibitor. For example, a LIGHT inhibitor variant or derivative may have less or greater activity than non-variant or non-derivatized LIGHT inhibitor.

Non-limiting examples of activities that can be retained, at least in part, include inhibitory or antagonist activity, binding affinity (e.g., K_{d}), avidity and binding selectivity (specificity) or non-selectivity. The variant or derivatized inhibitor can exhibit an activity (e.g., binding affinity) that is greater or less than a corresponding non-variant or non-derivatized inhibitor, e.g., greater or less inhibitory activity, binding affinity (e.g., K_{d}), avidity or binding selectivity (specificity) or non-selectivity. For example, “at least a part” of an activity of an inhibitor can be when the variant or derivatized agent has less of an inhibitory activity, e.g., 10-25%, 25-50%, 50-60%, 60-70%, 70-75%, 75-80%, 80-85%, 85-90%, 90-95%, 95-99%, 100%, or any percent or numerical value or range or value within such ranges. An activity of an inhibitor can be when the variant or derivatized agent has more inhibitory activity, e.g., 110-125%, 125-150%, 150-175%, 175-200%, 200-250%, 250-300%, 300-400%, 400-500%, 500-1000%, 1000-2000%, 2000-5000%, or more, or any percent or numerical value or range or value within such ranges. At least a part of binding affinity of an inhibitor can be when the variant or derivatized inhibitor has less affinity, e.g., 1-3-fold, 1-5-fold, 2-5 fold, 5-10-fold, 5-15-fold, 1015-fold, 15-20-fold, 20-25-fold, 25-30-fold, 30-50-fold, 50-100 fold, 100-500-fold 500-1000-fold, 1000-5000-fold, or less (e.g., K_{d}), or any numerical value or range of values within such ranges. At least a part of binding affinity of an inhibitor can be when the variant or derivatized inhibitor has more affinity, e.g., 1-3-fold, 1-5-fold, 2-5 fold, 5-10-fold, 5-15-fold, 10-15-fold, 15-20-fold, 20-25-fold, 25-30-fold, 30-50-fold, 50-100 fold, 100-500-fold 500-1000-fold, 1000-5000-fold, or more (e.g., K_{d}), or any numerical value or range of values within such ranges.

LIGHT inhibitors can be identified by assays known in the art. For example, the amount of activity can be assessed directly, such as measuring the particular activity (e.g., inhibitor activity, binding affinity, avidity, selectivity (specificity) or non-selectivity). For example, a LIGHT inhibitor can be identified by inhibition of HVEM or LTβR mediated lymphocyte activation or cell proliferation. A LIGHT inhibitor can also be identified by change in cell expression of a marker, such as ICAM expression. LIGHT inhibitors can further be identified by the ability to inhibit binding of purified LIGHT to purified HVEM or LTβR (or HVEM-IgG or LTβR-IgG fusion proteins), for example, when immobilized on a substrate (e.g., plastic) by ELISA, or when any of the molecules are transfected into cells that can be identified by labeling with the corresponding binding partner by flow cytometry. More particularly, for ELISA assays, plate bound LIGHT can be pre-incubated with LIGHT specific inhibitory molecules and blockade of receptor fusion protein binding measured by detection of the binding of the Fc fusion protein or lack of binding. Blockade of cell surface associated LIGHT binding to receptors is assessed by pre-incubation of LIGHT inhibitory molecules with cell lines expressing LIGHT on the surface followed by addition of receptor Fc fusion proteins. Assessment of inhibition is measured by detection of binding of the receptor fusion proteins or lack of binding by flow cytometry. Inhibition of LIGHT signaling in vitro can be determined by inhibiting LIGHT mediated chemokine secretion from colonic epithelial cells (HT29).

As used herein, the term “the same,” when used in reference to a LIGHT inhibitor means that the activity is within about 50% more than or less than the reference inhibitor. The term “substantially the same” when used in reference to inhibitor activity means that the activity is within about 100-500% (2-5-fold) or any percent value or range of percent values within such ranges, more than or less than the reference inhibitor. The same, when used in reference to binding affinity, means that the dissociation constant (K_{d}) is within about 1-5-fold, or any numerical value or range of values within such a range, of the referenced agent (e.g., 1-5 fold greater affinity or 1-5 fold less affinity than the reference agent).

The term “substantially the same” when used in reference to binding affinity, means that the dissociation constant (K_{d}) is within about 5 to 100 fold, or any numerical value or range of values within such a range, of the reference inhibitor (5-100 fold greater affinity or 5-100 fold less affinity than the reference inhibitor). The term “the same,” when used in reference to association constant (Kₐ) is within about 1 to 5 fold, or any numerical value or range of values within such a range, of the reference inhibitor (within 1-5 fold greater or 1-5 fold less than the association constant, Kₐ). The term “substantially the same” when used in reference to association constant (Kₐ), means that the association constant is within about 5 to 100 fold greater or less, or any numerical value or range of values within such a range, than the association constant, Kₐ, of the reference inhibitor (5-100 fold greater or 5-100 fold less than the reference inhibitor).

Dissociation (K_{d}) constants can be measured using radiolabeled inhibitors in competitive binding assays with increasing amounts of unlabelled inhibitor to generate saturation curves. The target, ligand or receptor used in the binding assay (e.g., LIGHT, HVEM, or LTβR, etc.) can be expressed in vitro, on cells or be present in extracts. Association (Kₐ) and dissociation (K_{d}) constants can be measured using surface plasmon resonance (SPR) (Rich and Myszka, Curr. Opin. Biotechnol. 11:54 (2000); Englebienne, Analyst. 123:1599 (1998)). SPR methods for real time detection and monitoring of protein binding rates are known and are commercially available and can be used to determine dissociation (Kₐ) constants (BiaCore 2000, Biacore AB, Upsala, Sweden; and Malmqvist, Biochem. Soc. Trans. 27:335 (1999)).

As used herein, the term “contact” and grammatical variations thereof means a physical or functional interaction between one entity and one or more other entities. An example of physical contact is a direct or indirect binding, such as between a LIGHT inhibitor and a target or receptor. An example of a functional interaction is where an intermediate facilitates or mediates a change in activity of one entity by another entity, such as a signaling pathway where molecules within the pathway functionally interact but need not physically contact each other. In the methods, contact can occur in solution, in solid phase, in vitro, ex vivo or in vivo (i.e., in a subject).

In accordance with the invention, there are provided methods in solution, in solid phase, in vitro, ex vivo or in vivo (i.e., in a subject). In one embodiment, a method includes contacting or administering to a subject, e.g. a subject in need thereof, an amount of a LIGHT inhibitor to treat the subject. In one particular aspect, an amount of LIGHT inhibitor contacted with or administered to the subject is sufficient to reduce or inhibit skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. In another particular aspect, an amount of LIGHT inhibitor contacted with or administered to the subject is sufficient to treat scleroderma, atopic dermatitis, nephrogenic fibrosing dermopathy, mixed connective tissue disease, scleromyxedema, scleredema, keloid, sclerodactyly, or eosinophilic fasciitis. In a further aspect, an amount of LIGHT inhibitor is administered to a subject sufficient to treat a skin fibrotic disease or disorder. In a still further aspect, an amount of LIGHT inhibitor is administered to a subject whom has previously experienced skin inflammation, skin fibrosis, skin fibrosis or a skin fibrotic disease or disorder, scleroderma, atopic dermatitis nephrogenic fibrosing dermopathy, mixed connective tissue disease, scleromyxedema, scleredema, keloid, sclerodactyly, or eosinophilic fasciitis, or is in need of treatment for or has been diagnosed with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis.

As used herein, the term “associated with,” when used in reference to the relationship between a symptom and a condition, disorder or disease, means that the symptom is caused by the referenced condition, disorder or disease, or is a secondary effect of the referenced condition, disorder or disease. A symptom that is present in a subject may therefore be the direct result of or caused by the referenced condition, or may be due at least in part to the subject reacting or responding to the referenced condition, disorder or disease, e.g., a secondary effect. For example, symptoms that occur as a result of skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis are due in part to hypersensitivity or an aberrant response of the immune system of the subject.

As used herein, the term “subject” includes animals, typically mammalian animals, such as but not limited to humans, non-human primates (apes, gibbons, chimpanzees, orangutans, macaques), domestic animals (dogs and cats), farm animals (horses, cows, goats, sheep, pigs), and experimental animals (mouse, rat, rabbit, guinea pig). Subjects include animal disease models (e.g., skin inflammation, skin fibrosis, scleroderma, or a skin fibrotic disease or disorder). Subjects include naturally occurring or non-naturally occurring mutated or non-human genetically engineered (e.g., transgenic or knockout) animals. Subjects further include animals having or at risk of having a chronic or acute condition, disorder or disease.

Conditions, disorders and diseases treatable in accordance with the invention include, for example, chronic or acute skin inflammation, skin fibrosis, scleroderma, or a skin fibrotic disease or disorder. An “inflammatory” condition, disorder or disease refers to one or more physiological responses that characterize or constitute inflammation.

In accordance with the invention, there are provided methods of reducing progression, severity, frequency, duration, susceptibility or probability of skin inflammation, skin fibrosis, skin fibrosis or a skin fibrotic disease or disorder, scleroderma, atopic dermatitis nephrogenic fibrosing dermopathy, mixed connective tissue disease, scleromyxedema, scleredema, keloid, sclerodactyly, or eosinophilic fasciitis. In one embodiment, a method includes administering to a subject an amount of LIGHT inhibitor sufficient to reduce or decrease progression, severity, frequency, duration, susceptibility or probability of one or more adverse symptoms associated with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis.

In another embodiment, a method includes administering to a subject an amount of LIGHT inhibitor sufficient to reduce or decrease progression, severity, frequency, duration, susceptibility or probability of one or more adverse symptoms caused by or associated with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. In one aspect, the adverse symptom is selected from skin inflammation or tissue damage; hardening or tightening of patches of skin; epidermis or dermis thickening; skin tenderness; skin itching; skin rash; heightened response or sensitivity to of skin to cold or hot temperatures; or numbness, pain or color changes in the fingers or toes. In yet another aspect, the subject has been diagnosed as having skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis.

Skin inflammation, skin fibrosis, scleroderma, or a skin fibrotic disease or disorder conditions include allergic and non-allergic skin inflammation, skin fibrosis, scleroderma, or a skin fibrotic disease or disorder, which may be provoked by a variety of factors including aberrant or undesirable immune responses. Alternatively or in addition to, skin inflammation, skin fibrosis, scleroderma, or a skin fibrotic disease or disorder may be caused by or associated with irritant particles (allergens such as pollen, dust, venoms, cotton, dander, foods). Skin inflammation, skin fibrosis, scleroderma, or a skin fibrotic diseases or disorders can be acute, chronic, mild, moderate or severe.

An “allergen” is a substance that can promote, stimulate or induce skin inflammation, skin fibrosis, scleroderma, atopic dermatitis or skin fibrotic diseases or disorders in a subject. Allergens include plant/tree pollens, insect venoms, animal dander, house dust mite, dust, fungal spores, latex, food and drugs (e.g., penicillin). Examples of particular allergens include proteins specific to the following genera: Canis (Canis familiaris); Dermatophagoides (e.g., Dermatophagoides farinae); Felis (Felis domesticus); Ambrosia (Ambrosia artemiisfolia); Lolium (e.g., Lolium perenne or Lolium multiflorum); Cryptomeria (Cryptomeria japonica); Alternaria(Alternariaalternata); Alder; Alnus (Alnusgultinosa); Betula (Betulaverrucosa); Quercus (Quercus alba); Olea (Oleaeuropa); Artemisia (Artemisia vulgaris); Plantago (e.g., Plantagolanceolata); Parietaria (e.g., Parietariaofficinalisor Parietariajudaica); Blattella (e.g., Blattellagermanica); Apis (e.g., Apismultiflorum); Cupressus (e.g., Cupressussempervirens, Cupressusarizonica and Cupressusmacrocarpa); Juniperus (e.g., Juniperussabinoides, Juniperusvirginiana, Juniperuscommunis and Juniperusashei); Thuya (e.g., Thuyaorientalis); Chamaecyparis (e.g., Chamaecyparisobtusa); Periplaneta (e.g., Periplanetaamericana); Agropyron (e.g., Agropyronrepens); Secale (e.g., Secalecereale); Triticum (e.g., Triticumaestivum); Dactylis(e.g., Dactylisglomerata); Festuca(e.g., Festucaelatior); Poa (e.g., Poapratensisor Poacompressa); Avena (e.g., Avena sativa); Holcus (e.g., Holcuslanatus); Anthoxanthum(e.g., Anthoxanthumodoratum); Arrhenatherum (e.g., Arrhenatherumelatius); Agrostis (e.g., Agrostis alba); Phleum (e.g., Phleumpratense); Phalaris (e.g., Phalarisarundinacea); Paspalum (e.g., Paspalumnotatum); Sorghum (e.g., Sorghum halepensis); and Bromus (e.g., Bromus inermis). Allergens also include peptides and polypeptides used in experimental animal models of allergy and asthma, including ovalbumin (OVA) and Schistosoma mansoni egg antigen.

A “skin fibrotic disease or disorder” means a condition, disorder or disease related to a skin or dermal tissue. Examples include, but are not limited to, scleroderma and atopic dermatitis.

Particular non-limiting examples of subjects include subjects having or at risk of having skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. Non-limiting examples of subjects further include subjects having or at risk of having an adverse or undesirable symptom associated with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. Such at risk subjects can be identified by a personal or family history, through genetic screening, tests appropriate for detection of increased risk, or exhibiting relevant symptoms indicating predisposition or susceptibility.

Subjects having or at risk of having skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis include subjects with an existing condition or a known or a suspected predisposition towards developing a symptom associated with or caused by skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. Thus, the subject can have active chronic or acute skin inflammation, skin fibrosis, or scleroderma, atopic dermatitis or other skin fibrotic disease or disorder or latent skin inflammation, skin fibrosis, scleroderma atopic dermatitis or other skin fibrotic disease or disorder. Thus, at risk subjects include those at risk from suffering from a condition based upon a prior personal or family history, and the season or physical location, but which the condition or a symptom associated with the condition may not presently manifest itself in the subject.

At risk subjects also appropriate for treatment in accordance with the invention include subjects susceptible to developing skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. At risk subjects appropriate for treatment in accordance with the invention include subjects having a predisposition towards skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis due to a genetic or environmental risk factor. Methods of the invention include subjects contacted with or administered to a binding agent prophylactically, e.g., prior to manifestation of skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis or a symptom thereof.

In the methods of the invention in which a detectable result or beneficial effect is a desired outcome, such as a therapeutic benefit in a subject treated in accordance with the invention, compositions such as LIGHT inhibitors can be administered in sufficient or effective amounts. An “amount sufficient” or “amount effective” includes an amount that, in a given subject, can have a desired outcome or effect. The “amount sufficient” or “amount effective” can be an amount of a LIGHT inhibitor that provides, in single or multiple doses, alone or in combination with one or more other (second) compounds or agents (e.g., a drug), treatments or therapeutic regimens, a long or short term detectable response, a desired outcome or beneficial effect in a particular given subject of any measurable or detectable degree or duration (e.g., for minutes, hours, days, months, years, or cured).

An amount sufficient or an amount effective can but need not be provided in a single administration and can but need not be administered alone (i.e., without a second drug, agent, treatment or therapeutic regimen), or in combination with another compound, agent, treatment or therapeutic regimen. In addition, an amount sufficient or an amount effective need not be sufficient or effective if given in single or multiple doses without a second compound, agent, treatment or therapeutic regimen, since additional doses, amounts or duration above and beyond such doses, or additional drugs, agents, treatment or therapeutic regimens may be included in order to be effective or sufficient in a given subject. Further, an amount sufficient or an amount effective need not be effective in each and every subject, nor a majority of subjects in a given group or population. Thus, as some subjects may not benefit from such treatments an amount sufficient or an amount effective means sufficiency or effectiveness in a particular subject, not a group or the general population. As is typical for such methods, some subjects will exhibit a greater or less response to a method of the invention, including treatment/therapy.

Reducing, inhibiting decreasing, eliminating, delaying, halting or preventing a progression or worsening or an adverse symptom of the condition, disorder or disease is a satisfactory outcome. The dose amount, frequency or duration may be proportionally increased or reduced, as indicated by the status of the condition, disorder or disease being treated, or any adverse side effects of the treatment or therapy. Dose amounts, frequencies or duration also considered sufficient and effective are those that result in a reduction of the use of another drug, agent, treatment or therapeutic regimen or protocol. For example, a LIGHT inhibitor is considered as having a beneficial or therapeutic effect if contact, administration or delivery in vivo results in the use of a lesser amount, frequency or duration of another drug, agent, treatment or therapeutic regimen or protocol to treat the condition, disorder or disease, or an adverse symptom thereof.

An “amount sufficient” or “amount effective” includes reducing, preventing, delaying or inhibiting onset, reducing, inhibiting, delaying, preventing or halting the progression or worsening of, reducing, relieving, alleviating the severity, frequency, duration, susceptibility or probability of one or more adverse or undesirable symptoms associated with the condition, disorder or disease of the subject. In addition, hastening a subject's recovery from one or more adverse or undesirable symptoms associated with the condition, disorder or disease is considered to be an amount sufficient or effective. Various beneficial effects and indicia of therapeutic benefit are as set forth herein and are known to the skilled artisan.

An “amount sufficient” or “amount effective,” in the appropriate context, can refer to therapeutic or prophylactic amounts. Therapeutically or prophylactically sufficient or effective amounts mean an amount that, in a given subject, detectably improves the condition, disorder or disease, such as an inflammatory condition, disorder or disease, as assessed by one or more objective or subjective clinical endpoints appropriate for the condition, disorder or disease.

In accordance with the invention, there are provided methods which provide a beneficial effect, such as a therapeutic benefit, to a subject. In one embodiment, a method includes administering an amount of LIGHT inhibitor sufficient to provide a therapeutic benefit or beneficial effect to a subject. In one aspect, a method reduces or inhibits probability, susceptibility, severity, frequency, duration or prevents skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis in the subject. In another aspect, a method reduces the probability, susceptibility, severity, frequency, duration or prevents skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis in the subject. In an additional aspect, a method reduces or inhibits probability, susceptibility, severity, frequency, duration or prevents a symptom caused by or associated with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis. In a further aspect, a method is sufficient to reduce progression, severity, frequency, duration, susceptibility, probability, halt, eliminate or prevent one or more adverse physiological or psychological symptoms associated with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis.

Sufficiency or effectiveness of a particular treatment can be ascertained by various clinical indicia and endpoints. An “amount sufficient” or “amount effective” is therefore an amount that provides an objective or subjective reduction or improvement in progression, severity, frequency, susceptibility or probability of skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis etc. Thus, a reduction, decrease, inhibition, delay, halt, prevention or elimination of one or more adverse symptoms of skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis can be used as a measure of sufficiency or effectiveness.

An “amount sufficient” or “amount effective” also includes an amount that, when used in combination with another binding agent, drug, or treatment or therapeutic regimen, reduces the dosage frequency, dosage amount, or an adverse symptom or side effect of the other binding agent, drug or treatment or therapeutic regimen, or eliminates the need for the other binding agent, drug or treatment or therapeutic regimen. For example, an “amount sufficient” or “amount effective” of a LIGHT inhibitor could result in a reduction in the dosage frequency or dosage amount of a steroid, antihistamine, beta adrenergic agonist, anticholinergic, methylxanthine, anti-IgE, anti-leukotriene, anti-beta2 integrin, anti-CCR3 antagonist, or anti-selectin required to achieve the same clinical endpoint.

The terms “treat,” “therapy” and grammatical variations thereof when used in reference to a method means the method provides an objective or subjective (perceived) improvement in a subjects' condition, disorder or disease, or an adverse symptom associated with the condition, disorder or disease. Non-limiting examples of an improvement can therefore reduce or decrease the probability, susceptibility or likelihood that the subject so treated will manifest one or more symptoms of the condition, disorder or disease. Additional symptoms and physiological or psychological responses caused by or associated with skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis are set forth herein and known in the art and, therefore, improvements in these and other adverse symptoms or physiological or psychological responses can also be included in the methods of the invention.

Methods of the invention therefore include providing a detectable or measurable beneficial effect or therapeutic benefit to a subject, or any objective or subjective transient or temporary, or longer-term improvement (e.g., cure) in the condition. Thus, a satisfactory clinical endpoint is achieved when there is an incremental improvement in the subjects condition or a partial reduction in the severity, frequency, duration or progression of one or more associated adverse symptoms or complications or inhibition, reduction, elimination, prevention or reversal of one or more of the physiological, biochemical or cellular manifestations or characteristics of the condition, disorder or disease. A therapeutic benefit or improvement (“ameliorate” is used synonymously) therefore need not be complete ablation of any or all adverse symptoms or complications associated with the condition, disorder or disease but is any measurable or detectable objectively or subjectively meaningful improvement in the condition, disorder or disease. For example, inhibiting a worsening or progression of the condition, disorder or disease, or an associated symptom (e.g., slowing or stabilizing one or more symptoms, complications or physiological or psychological effects or responses), even if only for a few days, weeks or months, even if complete ablation of the condition, disorder or disease, or an associated adverse symptom is not achieved is considered to be beneficial effect.

Prophylactic methods are included. “Prophylaxis” and grammatical variations thereof mean a method in accordance with the invention in which contact, administration or in vivo delivery to a subject is prior to manifestation or onset of a condition, disorder or disease (or an associated symptom or physiological or psychological response), such that it can eliminate, prevent, inhibit, decrease or reduce the probability, susceptibility, onset or frequency of having a condition, disorder or disease, or an associated symptom. Target subject's for prophylaxis can be one of increased risk (probability or susceptibility) of contracting the condition, disorder or disease, or an associated symptom, or recurrence of a previously diagnosed condition, disorder or disease, or an associated symptom, as set forth herein.

Any compound or agent (e.g., drug), therapy or treatment having a beneficial, additive, synergistic or complementary activity or effect (beneficial or therapeutic) can be used in combination with a binding agent in accordance with the invention. A “second compound” or “second agent” refers to any compound or agent (e.g., drug) that is not the first compound or agent of the recited composition, e.g., if a first drug or agent is a particular LIGHT inhibitor, then a second drug or agent is different from the first LIGHT inhibitor. The second compound or agent can but need not be selective, for example, for binding to LIGHT, HVEM or LTβR.

In accordance with the invention there are provided methods in which a second compound or agent (e.g., drug) is administered to the subject. In one embodiment, a second compound or agent (e.g., drug) is administered to the subject prior to, with or following contacting or administering a LIGHT inhibitor.

Methods of the invention therefore include combination therapies and treatments. Examples of such combination therapies include separate or pooled compounds or LIGHT inhibitors (e.g., pooled antagonists, compounds or agents). Accordingly, combination compositions, therapies and treatments are provided, as well as methods of using such combinations, therapies and treatments in conjunction with the methods of the invention. Contact, administration or in vivo delivery of a compound or agent, such as a binding agent, or practice of a therapy or treatment, can occur prior to, in conjunction with or following a method or method step of the invention, e.g., prior to, in conjunction or following administering a LIGHT inhibitor.

Non-limiting examples of functional classes of compounds and agents useful as a second compound or agent (e.g., drug) include anti-inflammatory, and anti-allergy drugs. Additional non-limiting examples of compounds and agents useful for employing in the invention, for example to treat skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis include hormones, such as steroids (e.g., glucocorticoids); antihistamines; beta adrenergic agonists; anticholinergics; methylxanthines; anti-IgE; anti-leukotrienes; anti-beta2 integrins; anti-alpha-4 integrins; H1-receptor antagonists; anti-CCR3 antagonists; and anti-selectins.

Specific non-limiting examples of glucocorticoids include dexamethasone, triamcinolone acetonide (AZMACORT®), beclomethasone, dipropionate (VANCERIL®), flunisolide (AEROBID®), fluticasone propionate (FLOVENT®), prednisone, methylprednisolone and mometasonefuroate (ASMANEX®, TWISTHALER®). Specific non-limiting examples of antihistamines include chlorcyclizine, chlorpheniramine, triprolidine (ACTIFED®), diphenhydramine hydrochloride (BENADRYL®), fexofenadine hydrochloride (ALLEGRA®), hydroxyzine hydrochloride (ATARAX®), loratadine (CLARITIN®), promethazine hydrochloride (PHENERGAN®), pyrilamine; and anti-IgE omalizumab (XOLAIR®). Specific non-limiting example of beta adrenergic agonists include albuterol (VENTOLIN®; PROVENTIL®), Xopenex®, (S)-isomer subtracted from racemic albuterol (Sepracor Inc.), pirbuterol, epinephrine, racepinephrine, adrenaline, isoproterenol, salmeterol (Serevent®), metaproterenol (ALUPENT®), bitolterol (Tornalate®), fenoterol (BEROTEC®), formoterol (Foradil®), isoetharine, procaterol, β2-adrenoceptor and terbutaline (BRETHINE®, LAMISIL®). A specific non-limiting example of an anticholinergic (cholinergic receptor antagonist) includes ipratropium bromide (ATROVENT®) and tiotropium. Specific non-limiting examples of methylxanthines include theophylline, aminophylline, theobromine, cromolyn (Intal®) and nedocromil (Fisons). A specific non-limiting example of an anti-IgE is omalizumab (XOLAIR®). Specific non-limiting examples of anti-leukotrienes (leukotriene inhibitors) include cysteinyl-leukotriene (Cys-LT), Singulair® and Accolate®.

Anti-inflammatory agents useful for employing in the methods include cytokines and chemokines. Particular non-limiting examples of cytokines include anti-inflammatory cytokines such as IL-4 and IL-10. Anti-cytokines and anti-chemokines, such as antibodies that bind to pro-inflammatory cytokines, TNFα, IFNγ, IL-1, IL-2, IL-6, etc., as well as anti-Th2 cytokines such as IL-5, IL-13, etc., can be employed in the methods.

Additional functional classes of compounds and agents useful as a second compound or agent (e.g., drug) include selective or non-selective potassium channel activators (bronchodilatators); muscarinic M3 receptor antagonists; M2 receptor agonists; opioid receptor agonists (inhibit release of sensory neuropeptides); H3-receptor agonists (inhibit acetylcholine release); phospholipase A2 inhibitors; 5-lipoxygenase inhibitors; 5-lipoxygenase activating protein (FLAP) inhibitors; phosphodiesterase inhibitors; immunomodulating agents (Ciclosporine); antibody against adhesion molecules; and antagonists of tachykinins (e.g., Substance P or neurokinin).

Compositions including LIGHT inhibitors can be included in a pharmaceutically acceptable carrier (excipient, diluent, vehicle or filling agent) for administration to a subject. The terms “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Exemplary antifungals include agents such as benzoic acid, undecylenicalkanolamide, ciclopiroxolamine, polyenes, imidazoles, allylamine, thicarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopiroxolamine, haloprogin, ketoconazole, undecylenate, silver sulfadiazine, undecylenic acid, undecylenicalkanolamide and Carbol-Fuchsin.

The pH can be adjusted by use or addition of pharmacologically acceptable acids or bases. Examples of inorganic acids include: hydrochloric acid, hydrobromic acid, nitric acid, sulfuric acid, and/or phosphoric acid. Examples of organic acids are: ascorbic acid, citric acid, malic acid, tartaric acid, maleic acid, succinic acid, fumaric acid, acetic acid, formic acid and/or propionic acid, etc. Acids which form an acid addition salt with the active ingredient may also be used. Examples of bases include alkali metal hydroxides and alkali metal carbonates. If such bases are used, the resulting salts which are contained in the pharmaceutical formulation, are typically compatible with the acid. If desired, mixtures of acids or bases may also be used.

Pharmaceutical compositions can optionally be formulated to be compatible with a particular route of administration. Thus, pharmaceutical compositions include carriers (excipients, diluents, vehicles or filling agents) suitable for administration by various routes and delivery to targets, topically, locally, regionally or systemically.

Exemplary routes of administration for contact or in vivo delivery which a composition can optionally be formulated include skin, dermis or epidermis, oral, buccal, intrapulmonary, intrauterine, intradermal, topical, dermal, parenteral, sublingual, subcutaneous, intravascular, intrathecal, intraarticular, intracavity, transdermal, iontophoretic, intraocular, ophthalmic, optical, intravenous, intramuscular, intraglandular, intraorgan, intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof.

Pharmaceutical compositions and delivery systems appropriate for compositions and methods of the invention are known to the skilled artisan (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315)

LIGHT inhibitors and pharmaceutical compositions thereof can be packaged in unit dosage form (capsules, troches, cachets, lozenges, or tablets) for ease of administration and uniformity of dosage. “Unit dosage form” as used herein refers to physically discrete units suited as dosages for treatment or therapy. Each unit contains a predetermined quantity of agent in association with the pharmaceutical carrier (excipient, diluent, vehicle or filling agent) which, when administered in one or more doses, is calculated to produce a desired beneficial effect. Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compositions for transdermal administration, such as “patches” adapted to remain in contact with the epidermis of the intended recipient for an extended or brief period of time. The individual unit dosage forms can be included in multi-dose kits or containers.

Dose amounts, frequency and duration for binding agents, including LIGHT inhibitors, or pro-drugs thereof, can be can be empirically determined in appropriate animal models. Dose amounts, frequency and duration can also be determined and optimized in human clinical trials.

The dosage amount can range from about 0.0001 mg/kg of subject body weight/day to about 1,000.0 mg/kg of subject body weight/day. Of course, doses can be more or less, as appropriate, for example, 0.00001 mg/kg of subject body weight to about 10,000.0 mg/kg of subject body weight, about 0.001 mg/kg, to about 1,000 mg/kg, about 0.01 mg/kg, to about 100 mg/kg, or about 0.1 mg/kg, to about 10 mg/kg of subject body weight over a given time period, e.g., 1, 2, 3, 4, 5 or more hours, days, weeks, months, years, in single bolus or in divided/metered doses.

As a non-limiting example, for treatment of skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis, a subject may be administered in single bolus or in divided/metered doses in the range of about 10 to 50,000 micrograms (“mcg”)/day, 10 to 20,000 mcg/day, 10 to 10,000 mcg/day, 25-1,000 mcg/day, 25 to 400 mcg/day, 25-200 mcg/day, 25-100 mcg/day or 25-50 mcg/day, which can be adjusted to be greater or less according to the weight of the subject, e.g., per pound, kilogram, etc.

LIGHT inhibitors, combinations of LIGHT inhibitors and other actives and pharmaceutical formulations thereof can be administered to a subject at any frequency, as a single bolus or in divided/metered doses, one, two, three, four or more times over a given time period, e.g., per hour, day, week, month or year. Exemplary dosage frequencies for skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis can vary, but are typically from 1-7 times, 1-5 times, 1-3 times, 2-times or once, daily, weekly or monthly, to reduce, inhibit, decrease, delay, prevent, halt or eliminate progression, severity, frequency, duration, or probability of one or more adverse symptoms of the conditions, disorders or diseases, as set forth herein or that would be apparent to one skilled in the art. Timing of contact, administration or in vivo delivery can be dictated by the condition, disorder or disease to be treated. For example, an amount can be administered to the subject substantially contemporaneously with, or within about 1-60 minutes or hours of the onset of a symptom associated with or caused by skin inflammation, skin fibrosis, skin fibrosis or a skin fibrotic disease or disorder, scleroderma, atopic dermatitis, nephrogenic fibrosing dermopathy, mixed connective tissue disease, scleromyxedema, scleredema, keloid, sclerodactyly, or eosinophilic fasciitis.

Dosage amount, frequency or duration can be increased, if necessary, or reduced, for example, once control of the condition, disorder or disease is achieved, dose amounts, frequency or duration can be reduced. Other conditions, disorders or diseases of the skin can be similarly treated, dosing amount, frequency or duration reduced, when adequate control of the condition, disorder or disease is achieved.

Of course, the dosage amount, frequency and duration can vary depending upon the judgment of the skilled artisan which will consider various factors such as whether the treatment is prophylactic or therapeutic, the type or severity of the condition, disorder or disease, the associated symptom to be treated, the clinical endpoint(s) desired such as the type and duration of beneficial or therapeutic effect. Additional non-limiting factors to consider in determining appropriate dosage amounts, frequency, and duration include previous or simultaneous treatments, potential adverse systemic, regional or local side effects, the individual subject (e.g., general health, age, gender, race, bioavailability), condition of the subject such as other disorders or diseases present and other treatments or therapies that the subject has or is undergoing (e.g., medical history). The skilled artisan will appreciate the factors that may influence the dosage, frequency and duration required to provide an amount sufficient to provide a subject with a beneficial effect, such as a therapeutic benefit.

The invention provides kits including LIGHT inhibitors suitable for practicing the methods, treatment protocols or therapeutic regimes herein, and suitable packing material. In one embodiment, a kit includes a LIGHT inhibitor, and instructions for administering or using the LIGHT inhibitor. In another embodiment, a kit includes a LIGHT inhibitor, an article of manufacture for delivery of the LIGHT inhibitor to the target area, organ, tissue or system (e.g., skin) and instructions for administering the LIGHT inhibitor.

The term “packing material” refers to a physical structure housing a component of the kit. The material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to a ampule, tube or vial containing a kit component. Labels or inserts can additionally include a computer readable medium, such as a disk (e.g., floppy diskette, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein (e.g., the binding agent or pharmaceutical composition), dose amounts, clinical pharmacology of the active agent(s) including mechanism of action, pharmacokinetics and pharmacodynamics. Labels or inserts can include information identifying manufacturer information, lot numbers, and location and date of manufacture.

Labels or inserts can include information on a condition, disorder or disease for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or therapeutic regimes described herein.

Labels or inserts can include information on any benefit that a component may provide, such as a therapeutic benefit. Labels or inserts can include information on potential adverse side effects, such as warnings to the subject or clinician regarding situations where it would not be appropriate to use a particular composition (e.g., a LIGHT inhibitor). For example, adverse side effects are generally more likely to occur at higher dose amounts, frequency or duration of the active agent and, therefore, instructions could include recommendations against higher dose amounts, frequency or duration. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities. Non-limiting examples of adverse side effects include, for example, hypersensitivity, rash, neurological effects such as tachycardia; palpitations; headache; tremor and nervousness.

Unless otherwise defined, all technical and scientific terms used herein have the same meaning as commonly understood by one of ordinary skill in the art to which this invention relates. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a LIGHT inhibitor” includes a plurality of LIGHT inhibitors; and reference to “a symptom” includes a plurality of symptoms (e.g., adverse or undesirable). Of course, this does not preclude limiting certain embodiments of the invention to specific LIGHT inhibitors or antagonists, particular symptoms, particular conditions, disorders or diseases, particular subjects, etc., using appropriate language.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 0-72 hrs, includes 1, 2, 3, 4, 5, 6, 7 hrs, etc., as well as 1, 2, 3, 4, 5, 6, 7 minutes, etc., and so forth. Reference to a range of doses, such as 0.1-1 ug/kg, 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, includes 0.11-0.9 ug/kg, 2-9 ug/kg, 11.5-24.5 ug/kg, 26-49 ug/kg, 55-90 ug/kg, 125-400 ug/kg, 750-800 ug/kg, 1.1-4.9 mg/kg, 6-9 mg/kg, 11.5-19.5 mg/kg, 21-49 mg/kg, 55-90 mg/kg, 125-200 mg/kg, 275.5-450.1 mg/kg, etc.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis disclosed herein. As an example, the invention includes embodiments in which specific subject matter disclosed herein is excluded from the embodiments. Thus, even though the invention is generally not expressed herein in terms of what the invention does not include, aspects that are not expressly included in the invention are nevertheless expressly or inherently disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

### Materials and Methods

### Mice

Six- to 8-week-old female WT, LIGHT^{-/-} (from Dr. K. Pfeffer (Scheu et al., 2002)), HVEM^{-/-} (from Dr. K. Pfeffer (Wang et al., 2005)) or TSLPR^{-/-} mice (from Dr. S. Ziegler (Carpino et al., 2004)) were bred in house on the C57BL/6 background. All experiments were in compliance with the regulations of the La Jolla Institute for Allergy and Immunology Animal Care Committee.

### Experimental Protocols

Mice were given 10 μg of recombinant mouse LIGHT (1794-LT/CF, R&D Systems) or PBS subcutaneously or intratracheally on days 1 and 2 and sacrificed one day later. Alternatively, mice were challenged with bleomycin (Sigma), 0.2 U/mouse, given intratracheally once, and monitored for skin inflammation and fibrosis after 7 or 14 days. For neutralization of LIGHT-LTβR interactions, mice were administered 200 μg of anti-LTβR mAb (LLTB2) (Anand et al., 2006) given i.v. one day prior to injection of bleomycin or rLIGHT and every other day until the end of the experiment. The anti-LTβR mAb (LLTB2) specifically blocks binding of LIGHT to LTβR (Anand et al., 2006). For neutralization of TGF-β, mice were administered 300 μg of anti-TGF-0 mAb (1D11, BioXCell) given i.p. one day prior to injection of rLIGHT and every other day thereafter.

### Quantitative Real-Time PCR

Total RNA was isolated using TRIzol (Invitrogen). To further purify RNA from skin samples, we used RNeasy Fibrous Tissue mini kit (Qiagen, 74704). Single-strand cDNA was prepared by reverse transcribing 5 μg of total RNA using Transcriptor First Strand cDNA kit (Roche). Samples were amplified in IQ SYRB Green Supermix (Bio-Rad Laboratories) using the primer pairs: LIGHT: forward, 5′-ACA GCC TTC AGT GTT TGT GGT G-3′; reverse, 5′-TCC GGT GGT TCT GTT CCA G-, Collagen: forward, 5′-GAG CCC TCG CTT CCG TAC TC-3′; reverse, 5′-TGT TCC CTA CTC AGC CGT CTG T-3′, aSMA: forward, 5′-TCT CTA TGC TAA CAA CGT CCT GTCA-3′; reverse, 5′-CCA CCG ATC CAG ACA GAG TAC TT-3′, TGF-β1: forward, 5′-CCC TAT ATT TGG AGC CTG GA-3′; reverse, 5′-GGA AGC TTC GGG ATT TAT GG-3′, muTSLP: forward, 5′-TCG AGG ACT GTG AGA GCA AGC CAG-3′; reverse, 5′-CTG GAG ATT GCA TGA AGG AAT ACC-3′, huTSLP: forward, 5′-TAT GAG TGG GAC CAA AAG TAC CG-3′; reverse, 5′-GGG ATT GAA GGT TAG GCT CTG G-3′. All samples were run in triplicate, and the mean values were used for quantification.

### Collagen and Skin Thickness Measurements

Formalin (4% in PBS) fixed skin sections were stained with Masson's Trichrome to evaluate collagen using an image analysis system (Image-Pro Plus; Media Cybernetics) (Cho et al., 2004). Epidermal thickness as well as dermal thickening was quantitated using Image Pro Analyser 7 software.

### Immunohistochemical Staining

Skin samples were de-paraffinized by sequential placement in xylene and ethanol. Sections were treated with Fc block in 10% Donkey serum (in PBS) and stained with (i) rabbit polyclonal antibody to alpha smooth muscle actin (clone 1A4, ab7817-abcam) at 1:200 concentration followed by Rhodamine Red-X AffiniPure Donkey anti-rabbit (Jackson Immunoresearch, 711295152) at 1:500; (ii) Goat polyclonal antibody to TSLP (clone L18, Santa Cruz Biotechnology) at 1:200 followed by Donkey anti-goat IgG-FITC (sc-2024, Santa Cruz Biotechnology) at 1:500. Slides were read on a Nikon 80i microscope and analyzed by LSM-Image software.

### Analysis of Skin Immune Cell Infiltrates

Cellular infiltration was assessed by flow on isolated skin cells using: CD45.2-BV711 (Clone 104; BD), CD3e-BV650 (Clone 145-2C11; BD), Mac3-Alexa Fluor 647 (Clone M4/84; Biolegend), Ly6-G/Ly6-G-Alexa Fluor 700 (Gr-1, clone RB6-8C5; Biolegend), SiglecF-PE-CF594 (Clone E50-2440; BD), CD11b-BB515 (Clone M1/70; BD), and CD11c-Brilliant Violet 785 (Clone N418; Biolegend). Macrophages were gated as CD45⁺, autofluorescent high, Mac3⁺, CD11c⁺ and SiglecF⁺; Dendritic cells as CD45⁺, CD11c⁺, CD11b⁺; Neutrophils as CD45⁺, GR1⁺, CD11b⁺ and Siglec F; T cells as CD45⁺, CD3⁺.

### Stimulation of Keratinocytes

Human Epidermal Keratinocytes from neonates (HEKn) (from Dr. Wendy Havran) or a mouse keratinocyte cell line, PAM212, were stimulated with 20 ng/ml of recombinant human LIGHT (R&D, 664-LI/CF) or mouse LIGHT for 72 h in Epilife media (Life technologies). TSLP was measured by immunostaining, using anti-hTSLP mAb (clone AF1398, from R&D) or anti-mTSLP (clone 18, Santa Cruz Biotechnology), and qPCR analyses. For TGF-β neutralization, 30 μg/ml of anti-TGF-β mAb (1D11) or its Isotype control were added into culture. LIGHT receptors were visualized using anti-LTβR and anti-HVEM (Biolegend).

### Statistical Analyses

Statistical analysis was performed using GraphPad Prism software. A nonparametric t test or Mann-Whitney test was used where indicated. A P value<0.05 was considered statistically significant.

### Example 2

### Soluble Recombinant LIGHT Induces Features of Skin Fibrosis

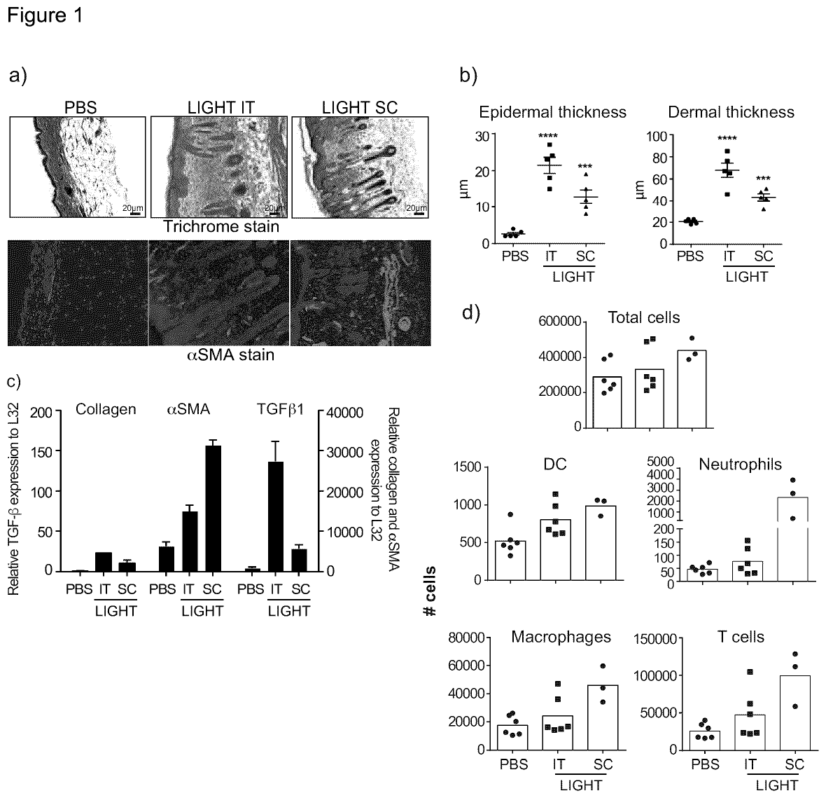

To determine whether LIGHT was able to drive fibrotic features in the skin, 10 μg of recombinant soluble murine LIGHT or PBS was injected twice, either subcutaneously (SC) or intratracheally (IT), with the notion for the latter that LIGHT would become systemic and possibly lead to activity in the skin. Collagen deposition, as assessed with Masson's trichrome stain, was observed in the skin penetrating into the adipose layer, and epidermal activity was obvious with pronounced keratinocyte hypertrophy (FIG. 1a). In line with this, we found that total skin collagen, as assessed by sircol assay, was increased by an average of 34% in mice injected with LIGHT when compared to mice injected with PBS. Similar results were seen regardless of the route of LIGHT administration (FIGS. 1a and 1b). Staining of alpha smooth muscle actin (aSMA) revealed a significant increase by basal keratinocytes and other cells that were likely myofibroblasts, again regardless of the mode of injection of LIGHT. Some vascular changes were observed with thickening of the endothelial wall of skin blood vessels, also highlighted by aSMA staining (FIG. 1a). Moreover, qPCR analysis confirmed up-regulation of collagen and aSMA transcripts, as well as TGF-β1 that is often associated with fibrotic activity, with both SC and IT injection (FIG. 1c). We additionally observed immune cellular infiltration into the skin, including dendritic cells, neutrophils, macrophages, and T cells, more prominent when LIGHT was given subcutaneously (FIG. 1d).

### Example 3

### Reduced Skin Fibrotic Activity in LIGHT-Deficient Mice

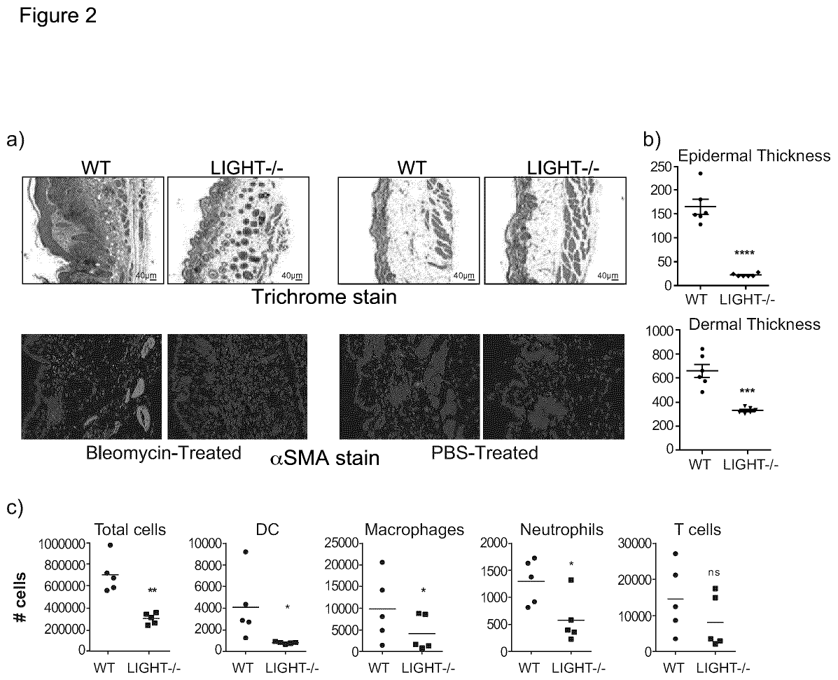

To determine whether endogenous LIGHT activity could contribute to fibrotic features in the skin, we used an established model with the antibiotic bleomycin that produces symptoms in mouse skin that have features in common with skin of patients with scleroderma (Yamamoto, 2006; Yamamoto and Nishioka, 2005). Interestingly, the phenotype in the skin of WT mice promoted by bleomycin was histologically similar to that induced by injection of recombinant LIGHT. Importantly, bleomycin-treated LIGHT^{-/-} mice failed to accumulate significant amounts of collagen with little evidence of trichrome stain in the sub-dermal adipose layer (FIG. 2a). Total skin collagen, based on sircol assay, was increased with a mean of 38% in WT mice given bleomycin over those given PBS, whereas a mean increase of only 13% was seen in LIGHT^{-/-} mice. Alpha smooth muscle actin expression was also not upregulated to any great extent in LIGHT^{-/-} mice (FIG. 2a). Moreover, quantitation of both dermal and epidermal thickening revealed a very pronounced activity of bleomycin in WT mice with markedly less in LIGHT^{-/-} mice (FIG. 2b). Cellular infiltration was additionally reduced in LIGHT-deficient mice compared to WT mice with an overall trend to fewer dendritic cells, macrophages, neutrophils, and T cells (FIG. 2c).

### Example 4

### Both LTβR and HVEM Contribute to Skin Fibrosis and Thickening

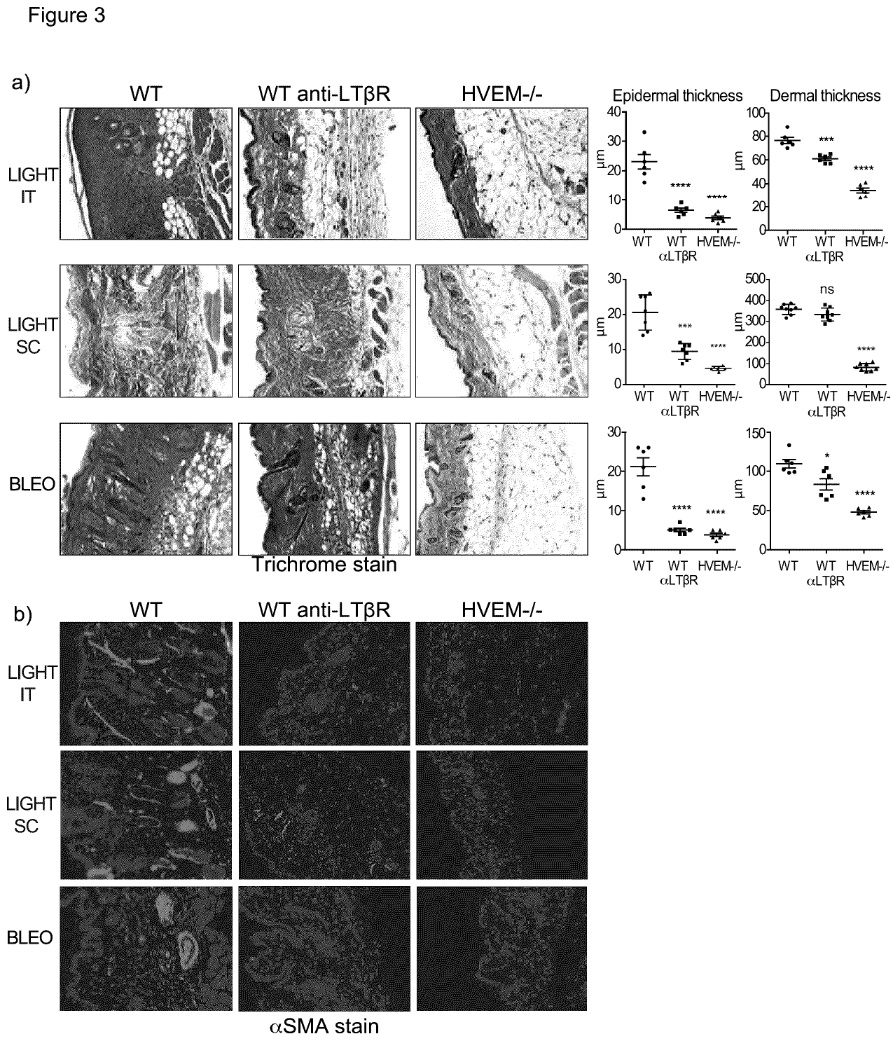

LIGHT binds to two receptors, LTβR/TNFRSF3 and HVEM/TNFRSF14. To understand whether one or both of these molecules were involved, we assessed responses in WT mice treated with an antibody that selectively neutralizes LIGHT-LTβR interactions and also tested responses in HVEM^{-/-} mice. In naïve mice injected with recombinant LIGHT, epidermal thickening and hypertrophy of keratinocytes was almost completely abrogated with deletion of HVEM or with blocking LTβR. In contrast, dermal fibrosis was reduced to a much greater extent in the absence of HVEM interactions. Blocking LIGHT-LTβR still reduced dermal and sub-dermal collagen slightly but to a markedly lesser degree (FIG. 3a). aSMA expression was also reduced when either LIGHT-HVEM or LIGHT-LTβR interaction was neutralized (FIG. 3b). Importantly, almost identical results were obtained in mice treated with bleomycin with epidermal thickening and aSMA accumulation being regulated by both LTβR and HVEM, and dermal thickening more dependent on HVEM (FIGS. 3a and 3b).

### Example 5

### LIGHT Upregulates TSLP In Vivo to Promote Skin Fibrosis

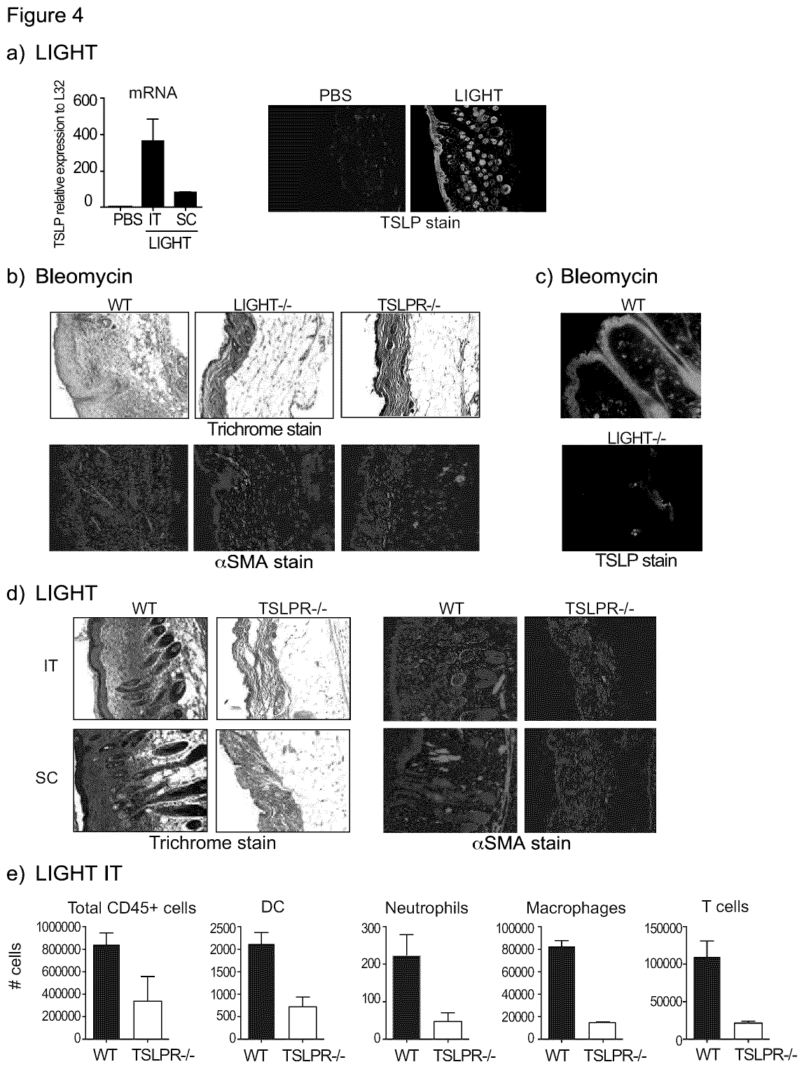

Given the role suggested for TSLP in promoting skin fibrosis when over-expressed in transgenic mice (Yoo et al., 2005b) and data that found TSLPR−/− mice did not develop pronounced skin fibrosis induced by subcutaneous injection of bleomycin (Usategui et al., 2013), we explored whether LIGHT might control TSLP expression. Significantly, in naïve mice injected with rLIGHT we found upregulated expression of TSLP mRNA within 3 days in the skin (FIG. 4a). Again, this was regardless of administration SC or IT. Importantly, induction of TSLP protein was easily visualized by immunohistochemistry in the skin, largely expressed in keratinocytes in both the epidermis and hair follicles (FIG. 4a). To extend this, we found a similar lack of the skin fibrotic phenotype in TSLPR−/− mice as observed in LIGHT−/− mice (FIG. 4b), and found a dramatic reduction in TSLP protein in the skin of bleomycin-treated LIGHT−/− mice (FIG. 4c). It was further demonstrated that essentially all of the hallmarks of disease, including collagen deposition and dermal and epidermal thickening, as well as aSMA accumulation and cellular infiltration, were abrogated when LIGHT was injected into TSLPR−/− mice (FIGS. 4d and 4e).

### Example 6

LIGHT Synergizes with TGF-β In Vivo to Promote TSLP Expression and Skin Fibrosis

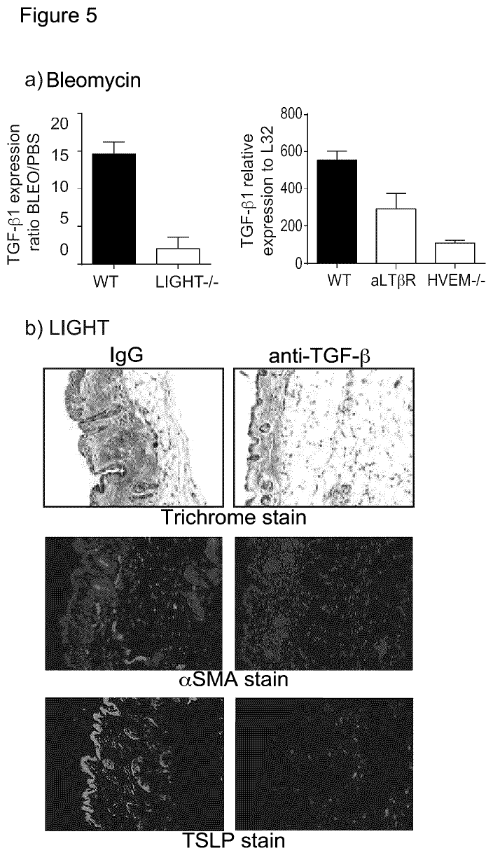

Recombinant LIGHT promoted TGF-β expression in the skin (FIG. 1). In accordance, we also found reduced TGF-β mRNA levels in the skin of LIGHT−/− mice, WT mice treated with anti-LTβR, and HVEM−/− mice, injected with bleomycin (FIG. 5a). As this cytokine has been implicated in fibrotic activity in many situations, we then neutralized TGF-β signaling in vivo. We observed a significant reduction in collagen deposition and aSMA accumulation, as well as interestingly a lower level of TSLP expression in the skin (FIG. 5b). This suggests that LIGHT and TGF-β synergized to promote TSLP, or LIGHT indirectly promoted TSLP through TGF-β.

### Example 7

### LIGHT Directly Induces TSLP in Keratinocytes Independently of TGF-β

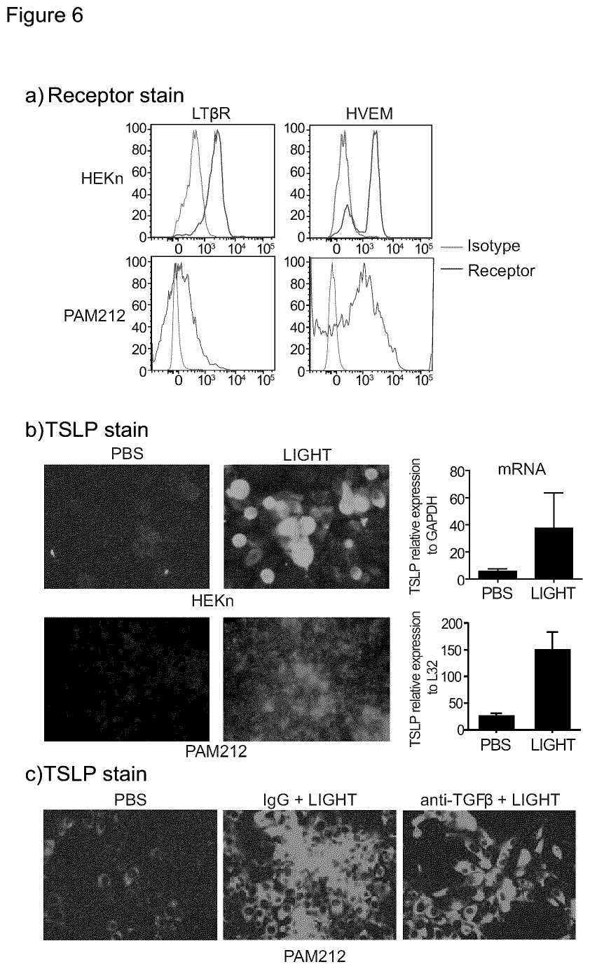

Because the primary source of TSLP in the skin appeared to be keratinocytes, we then asked whether human and mouse keratinocytes expressed the receptors for LIGHT and might directly respond by producing TSLP. Significantly, LTβR and HVEM were constitutively expressed on normal human epidermal keratinocytes and a mouse keratinocyte cell line (FIG. 6a). Furthermore, recombinant human LIGHT or mouse LIGHT strongly induced the expression of TSLP mRNA in the human and mouse keratinocytes, and TSLP protein was also detected by immunohistochemistry (FIG. 6b). Moreover, blocking TGF-β in these cultures did not ablate TSLP expression by keratinocytes suggesting a direct action (FIG. 6c). Lastly, as LIGHT promoted epidermal thickening in vivo, we asked if it could stimulate keratinocyte proliferation. However, BrdU and 7AAD analysis of keratinocytes stimulated with LIGHT did not reveal enhanced proliferation or any obvious difference in apoptosis (data not shown). As data in TSLPR−/− mice and with blocking TGF-β showed a lack of epidermal thickening, this suggests that LIGHT primarily acted indirectly through these cytokines to drive hypertrophy of keratinocytes in vivo.

### Example 8

### Discussion of Results

Disclosed herein is data demonstrating that LIGHT as a soluble molecule that very rapidly induced a fibrotic phenotype in the skin even in the absence of any other stimulus, and can promote the scleroderma- or atopic dermatitis-like phenotype that is characteristic of skin disease driven by bleomycin. Moreover, LIGHT controlled the production of TSLP and TGF-β in the skin and directly induced TSLP expression by keratinocytes.

TSLP was reported to be a central regulator or inducer of allergic inflammation, and a strong role of TSLP in the generation of Th2-type responses has been documented in many models (Comeau and Ziegler, 2010; Ziegler, 2010; Ziegler and Artis, 2010). The primary sources of TSLP are thought to be epithelial cells, and in line with this, transgenic mice where TSLP was over-expressed in bronchial epithelium or in keratinocytes exhibited tissue remodeling in those organs (Yoo et al., 2005b; Zhou et al., 2005). Furthermore, neutralization of TSLP signaling in a model of atopic dermatitis also resulted in a strong reduction in skin fibrosis (Zhu et al., 2011) and the data here and elsewhere (Usategui et al., 2013) show its important role in promoting skin fibrosis triggered by bleomycin. The finding disclosed herein that LIGHT-deficient mice displayed defective TSLP expression, and that TSLP can be directly upregulated in keratinocytes by LIGHT, provides evidence of the importance of LIGHT in driving or maintaining symptoms of fibrosis in the skin.

For LIGHT to be involved in a fibrotic response in the skin, its receptors need to be expressed. T cells, dendritic cells, and stromal cells express either HVEM and/or LTβR. However, most relevant to fibrosis in the skin may be either resident immune cells, such as dermal dendritic cells, macrophages, and fibroblasts, or as we focus on in this study keratinocytes. The identification of TSLP as a downstream product of LIGHT activity in keratinocytes, and TSLP as a major mediator of the profibrotic effects of LIGHT in vivo, strongly suggest there will be a central role of LTβR or HVEM expressed in the epidermis in contributing to the clinical manifestations of skin fibrosis. The results herein also disclose that LIGHT additionally promoted TGF-β expression in the skin, and that TGF-β was essential for the fibrotic activity of LIGHT as well as contributing to the expression of TSLP in vivo. However, LIGHT induction of TSLP from keratinocytes was TGF-β independent, suggesting another source of TGF-β is likely to synergize with LIGHT in vivo.

Similar to many molecules in the TNF superfamily, LIGHT is not constitutively expressed. It was first described as a product of activated T cells (Mauri et al., 1998), but has also been found to be made by NK cells, dendritic cells, and macrophages in some conditions (Ware, 2005, 2009). LIGHT might also be produced by other as yet unidentified cells. Bleomycin induces tissue injury and has been shown to induce modification of self-antigens, such as cleavage of topoisomerase I (Yamamoto, 2006, 2009; Yamamoto and Nishioka, 2005). Moreover, apoptosis is detected in the skin of human systemic sclerosis patients associated with the severity of tissue damage (Yamamoto, 2009; Yamamoto and Nishioka, 2005; Yoshizaki et al., 2010). Therefore, many of the above immune cell types could be activated to produce LIGHT during the development of a skin inflammatory response.

In summary, LIGHT is disclosed herein to promote fibrotic activity in the skin, which is dependent on both TSLP and TGF-β. Keratinocytes express both receptors for LIGHT, and that LIGHT can directly stimulate these cells to produce TSLP. The data herein indicates that LIGHT is an attractive target for therapy of disorders and diseases of the skin that are characterized by inflammation and/or fibrosis, such as scleroderma and atopic dermatitis.

### REFERENCES

Alysandratos K D, Angelidou A, Vasiadi M, et al. (2010) Increased affected skin gene expression and serum levels of thymic stromal lymphopoietin in atopic dermatitis. Ann Allergy Asthma Immunol 105:403-4.
Anand S, Wang P, Yoshimura K, et al. (2006) Essential role of TNF family molecule LIGHT as a cytokine in the pathogenesis of hepatitis. Journal of Clinical Investigation 116:1045-51.
Boin F, Wigley F (2009) Connective tissue diseases: Immunosuppressive therapy in SSc: what is the target? Nat Rev Rheumatol 5:357-8.
Carpino N, Thierfelder W E, Chang M S, et al. (2004) Absence of an essential role for thymic stromal lymphopoietin receptor in murine B-cell development. Mol Cell Biol 24:2584-92.
Cho J Y, Miller M, Baek K J, et al. (2004) Inhibition of airway remodeling in IL-5-deficient mice. Journal of Clinical Investigation 113:551-60.
Coda A B, Sinha A A (2011) Integration of genome-wide transcriptional and genetic profiles provides insights into disease development and clinical heterogeneity in alopecia areata. Genomics 98:431-9.
Comeau M R, Ziegler S F (2010) The influence of TSLP on the allergic response. Mucosal Immunol 3:138-47.
Croft M, Benedict C A, Ware C F (2013) Clinical targeting of the TNF and TNFR superfamilies. Nat Rev Drug Discov 12:147-68.
Doherty T A, Soroosh P, Khorram N, et al. (2011) The tumor necrosis factor family member LIGHT is a target for asthmatic airway remodeling. Nat Med 17:596-603.
Gao P S, Rafaels N M, Mu D, et al. (2010) Genetic variants in thymic stromal lymphopoietin are associated with atopic dermatitis and eczema herpeticum. J Allergy Clin Immunol 125:1403-7 e4.
Ishida S, Yamane S, Ochi T, et al. (2008) LIGHT induces cell proliferation and inflammatory responses of rheumatoid arthritis synovial fibroblasts via lymphotoxin beta receptor. J Rheumatol 35:960-8.
Kotani H, Masuda K, Tamagawa-Mineoka R, et al. (2012) Increased plasma LIGHT levels in patients with atopic dermatitis. Clinical and experimental immunology 168:318-24.
Lee E B, Kim K W, Hong J Y, et al. (2010) Increased serum thymic stromal lymphopoietin in children with atopic dermatitis. Pediatr Allergy Immunol 21:e457-60.
Mackay F, Majeau G R, Hochman P S, et al. (1996) Lymphotoxin beta receptor triggering induces activation of the nuclear factor kappaB transcription factor in some cell types. J Biol Chem 271:24934-8.
Mauri D N, Ebner R, Montgomery R I, et al. (1998) LIGHT, a new member of the TNF superfamily, and lymphotoxin alpha are ligands for herpesvirus entry mediator. Immunity 8:21-30.
Miyagaki T, Sugaya M, Suga H, et al. (2012) Low herpesvirus entry mediator (HVEM) expression on dermal fibroblasts contributes to a Th2-dominant microenvironment in advanced cutaneous T-cell lymphoma. J Invest Dermatol 132:1280-9.
Oh M H, Oh S Y, Yu J, Myers A C, et al. (2011) IL-13 induces skin fibrosis in atopic dermatitis by thymic stromal lymphopoietin. J Immunol 186:7232-42.
Pierer M, Brentano F, Rethage J, et al. (2007) The TNF superfamily member LIGHT contributes to survival and activation of synovial fibroblasts in rheumatoid arthritis. Rheumatology (Oxford) 46:1063-70.
Rosenbloom J, Castro S V, Jimenez S A (2010) Narrative review: fibrotic diseases: cellular and molecular mechanisms and novel therapies. Ann Intern Med 152:159-66.
Sano Y, Masuda K, Tamagawa-Mineoka R, et al. (2013) Thymic stromal lymphopoietin expression is increased in the horny layer of patients with atopic dermatitis. Clinical and experimental immunology 171:330-7.
Scheu S, Alferink J, Potzel T, et al. (2002) Targeted disruption of LIGHT causes defects in costimulatory T cell activation and reveals cooperation with lymphotoxin beta in mesenteric lymph node genesis. Journal of ExperimentalMedicine 195:1613-24.
Shikotra A, Choy D F, Ohri C M, et al. (2012) Increased expression of immunoreactive thymic stromal lymphopoietin in patients with severe asthma. J Allergy Clin Immunol 129:104-11 el-9.
Usategui A, Criado G, Izquierdo E, et al. (2013) A profibrotic role for thymic stromal lymphopoietin in systemic sclerosis. Ann Rheum Dis 72:2018-23.
Wang Y, Subudhi S K, Anders R A, et al. (2005) The role of herpesvirus entry mediator as a negative regulator of T cell-mediated responses. Journal of Clinical Investigation 115:711-17.
Ware C F (2005) Network communications: lymphotoxins, LIGHT, and TNF. Annual Review of Immunology 23:787-819.
Ware C F (2009) Targeting the LIGHT-HVEM pathway. Adv Exp MedBiol 647:146-55.
Wynn T A, Ramalingam T R (2012) Mechanisms of fibrosis: therapeutic translation for fibrotic disease. Nat Med 18:1028-40.
Yamamoto T (2006) The bleomycin-induced scleroderma model: what have we learned for scleroderma pathogenesis? Arch Dermatol Res 297:333-44.
Yamamoto T (2009) Scleroderma—pathophysiology. Eur J Dermatol 19:14-24.
Yamamoto T, Nishioka K (2005) Cellular and molecular mechanisms of bleomycin-induced murine scleroderma: current update and future perspective. Exp Dermatol 14:81-95.
Yao W, Zhang Y, Jabeen R, et al. (2013) Interleukin-9 is required for allergic airway inflammation mediated by the cytokine TSLP. Immunity 38:360-72.
Ying S, O'Connor B, Ratoff J, et al. (2008) Expression and cellular provenance of thymic stromal lymphopoietin and chemokines in patients with severe asthma and chronic obstructive pulmonary disease. J Immunol 181:2790-8.
Yoo J, Omori M, Gyarmati D, et al. (2005a) Spontaneous atopic dermatitis in mice expressing an inducible thymic stromal lymphopoietin transgene specifically in the skin. J Exp Med 202:541-9.
Yoo J, Omori M, Gyarmati D, et al. (2005b) Spontaneous atopic dermatitis in mice expressing an inducible thymic stromal lymphopoietin transgene specifically in the skin. Journal of Experimental Medicine 202:541-9.
Yoshizaki A, Yanaba K, Iwata Y, et al. (2010) Treatment with rapamycin prevents fibrosis in tight-skin and bleomycin-induced mouse models of systemic sclerosis. Arthritis Rheum 62:2476-87.
Zhou B, Comeau M R, De Smedt T, et al. (2005) Thymic stromal lymphopoietin as a key initiator of allergic airway inflammation in mice. Nature immunology 6:1047-53.
Zhu Z, Oh M H, Yu J, et al. (2011) The Role of TSLP in IL-13-induced atopic march. Sci Rep 1:23.
Ziegler S F (2010) The role of thymic stromal lymphopoietin (TSLP) in allergic disorders. Curr Opin Immunol 22:795-9.
Ziegler S F, Artis D (2010) Sensing the outside world: TSLP regulates barrier immunity. Nature immunology 11:289-93.

1.-43. (canceled)

44. A method of treating atopic dermatitis, comprising administering a sufficient amount of an inhibitor of LIGHT (p30 polypeptide) to a subject in need thereof to reduce or inhibit the atopic dermatitis in the subject.

45. The method of claim 44, wherein a Th1, Th9, or Th17 inflammatory response in skin is reduced or inhibited.

46. The method of claim 44, wherein the method reduces or inhibits progression, severity, frequency, duration or probability of a symptom of atopic dermatitis.

47. The method of claim 44, wherein one or more symptoms of atopic dermatitis is reduced, inhibited, abrogated, eliminated, or reversed.

48. The method of claim 47, wherein the one or more symptoms comprise skin inflammation or tissue damage; hardening or tightening of patches of skin; thickening of the dermis or epidermis; skin tenderness; skin itching; skin rash; heightened response or sensitivity to of skin to cold or hot temperatures; or numbness, pain or color changes in the fingers or toes.

49. The method of claim 47, wherein the one or more symptoms comprise infiltration of eosinophils and/or neutrophils in skin, leukocyte infiltration of skin, inflammation of skin, or increased Th1, Th2, Th9 or Th17 cytokine production.

50. The method of claim 49, wherein the cytokine is TSLP, TGF-beta or an interleukin (IL).

51. The method of claim 50, wherein the interleukin (IL) comprises IL-4, IL-5, IL-9, IL-13, IL-16, IL-17 or IL-25.

52. The method of claim 44, wherein the atopic dermatitis is caused by an allergen.

53. The method of claim 44, wherein the atopic dermatitis is not caused by an allergen.

54. The method of claim 44, wherein the atopic dermatitis is chronic or acute.

55. The method of claim 44, wherein the method reduces or decreases undesirable or abnormal eosinophil migration, chemotaxis or generation in skin.

56. The method of claim 44, wherein the inhibitor of LIGHT comprises an TβR-IgG fusion polypeptide.

57. The method of any of claim 44, further comprising contacting or administering a second drug to the subject prior to, with or following administering the chimeric LTβR polypeptide comprising a LTβR polypeptide sequence and an immunoglobulin sequence.

58. The method of claim 57, wherein the second drug comprises an anti-skin inflammation, anti-skin fibrosis, anti-scleroderma, or anti-skin fibrotic disease or disorder drug.

59. The method of claim 57, wherein the second drug comprises a hormone or a steroid.

60. The method of claim 44, wherein the subject is a human.
